(19) United States
(12) Patent Application Publication
(10) Pub. No.: US 2025/0360236 A1
(43) Pub. Date: Nov. 27, 2025

(54) PROSTATE SPECIFIC MEMBRANE ANTIGEN (PSMA) LIGANDS

(71) Applicants: Deutsches Krebsforschungszentrum Stiftung des öffentlichen Rechts, Heidelberg (DE); Albert-Ludwigs-Universität Freiburg, Freiburg (DE)

(72) Inventors: Matthias EDER, Heidelberg (DE); Ann-Christin EDER, Simonswald (DE)

(73) Assignees: Deutsches Krebsforschungszentrum Stiftung des öffentlichen Rechts, Heidelberg (DE); Albert-Ludwigs-Universität Freiburg, Freiburg (DE)

(21) Appl. No.: 18/866,052

(22) PCT Filed: May 16, 2023
(86) PCT No.: PCT/EP2023/063104

§ 371 Date: Nov. 14, 2024

(30) Foreign Application Priority Data
May 17, 2022 (EP) 22173812.3

(51) Int. Cl. A61K 51/04; A61K 49/00; A61P 35/00; C07D 403/14

(52) U.S. Cl. CPC A61K 51/0497; A61K 49/0032; A61K 51/0431; A61P 35/00; C07D 403/14; A61K 2121/00; A61K 2123/00

(57) ABSTRACT

The present invention generally relates to the field of dye labelled, preferably fluorescent dye labelled, radiopharmaceuticals and their use in nuclear medicine as tracers, imaging agents and for the treatment of various disease states of PSMA-expressing cancers, especially prostate cancer, and metastases thereof as well as their use in preoperative PET Imaging and Fluorescence-Guided Surgery of cancers, especially prostate cancer, and metastases thereof.

### FIELD OF THE INVENTION

The present invention generally relates to the field of dye labelled, preferably fluorescent dye labelled, radiopharmaceuticals and their use in nuclear medicine as tracers, imaging agents and for the treatment of various disease states of PSMA-expressing cancers, especially prostate cancer, and metastases thereof as well as their use in preoperative PET Imaging and Fluorescence-Guided Surgery of cancers, especially prostate cancer, and metastases thereof.

### RELATED ART

Prostate cancer (PCa) is the leading cancer in the US and European population. At least 1-2 million men in the western hemisphere suffer from prostate cancer and it is estimated that the disease will strike one in six men between the ages of 55 and 85. There are more than 300,000 new cases of prostate cancer diagnosed each year in USA. The mortality from the disease is second only to lung cancer. Currently, imaging methods with high resolution of the anatomy, such as computed tomography (CT), magnetic resonance (MR) imaging and ultrasound, predominate for clinical imaging of prostate cancer. An estimated annual $2 billion is currently spent worldwide on surgical, radiation, drug therapy and minimally invasive treatments of prostate cancer. For treatment of localized prostate cancer, radical prostatectomy with lymph node dissection is an established curative strategy. However, the precise localization and delineation of tumor margins and metastases remain challenging. There is presently no effective therapy for relapsing, metastatic, androgen-independent prostate cancer.

It is well known that tumors may express unique proteins associated with their malignant phenotype or may over-express normal constituent proteins in greater number than normal cells. The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins provide an attractive route for imaging and treating tumors under non-invasive conditions. A promising new series of low molecular weight imaging agents targets the prostate-specific membrane antigen (PSMA) (Mease R. C. et al. Clin Cancer Res. 2008, 14, 3036-3043; Foss, C. A.; et al. Clin Cancer Res 2005, 11, 4022-4028; Pomper, M. G.; et al. Mol Imaging 2002, 1, 96-101; Zhou, J.; et al. Nat Rev Drug Discov 2005, 4, 015-1026; WO 2013/022797).

A variety of experimental low molecular weight PCa imaging agents are currently being pursued clinically, including radiolabeled choline analogs [¹⁸F]fluorodihydrotestosterone ([¹⁸F]FDHT), anti-I-amino-3-[¹⁸F]fluorocycIobutyl-1-carboxylic acid (anti[¹⁸F]F-FACBC, [¹¹C]acetate and 1-(2-deoxy-2-[¹⁸F]flouro-L-arabinofuranosyl)-5-methyluracil ([¹⁸F]FMAU)(Scher, B.; et al. Eur J Nucl Med Mol Imaging 2007, 34, 45-53; Rinnab, L; et al. BJU Int 2007, 100, 786, 793; Reske, S. N.; et al. J Nucl Med 2006, 47, 1249-1254; Zophel, K.; Kotzerke, J. Eur J Nucl Med Mol Imaging 2004, 31, 756-759; Vees, H.; et al. BJU Int 2007, 99, 1415-1420; Larson, S. M.; et al. J Nucl Med 2004, 45, 366-373; Schuster, D. M.; et al. J Nucl Med 2007, 48, 56-63; Tehrani, O. S.; et al. J Nucl Med 2007, 48, 1436-1441). Each operates by a different mechanism and has certain advantages, e.g., low urinary excretion for [¹¹C]choline, and disadvantages, such as the short physical half-life of positron-emitting radionuclides.

PSMA is a trans-membrane, 750 amino acid type II glycoprotein that has abundant and restricted expression on the surface of PCa, particularly in androgen-independent, advanced and metastatic disease (Schulke, N.; et al. Proc Natl Acad Sci USA 2003, 100, 12590-12595). The latter is important since almost all PCa become androgen independent over the time. PSMA possesses the criteria of a promising target for therapy (Schulke, N.; et al. Proc. Natl. Acad. Sci. USA 2003, 100, 12590-12595). The PSMA gene is located on the short arm of chromosome 11 and functions both as a folate hydrolase and neuropeptidase. It has neuropeptidase function that is equivalent to glutamate carboxypeptidase II (GCPII), which is referred to as the “brain PSMA”, and may modulate glutamatergic transmission by cleaving/V-acetylaspartylglutamate (NAAG) to N-acetylaspartate (NAA) and glutamate (Nan, F.; et al. J Med Chem 2000, 43, 772-774). There are up to 10⁶ PSMA molecules per cancer cell, further suggesting it as an ideal target for imaging and therapy with radionuclide-based techniques (Tasch, J.; et al. Crit Rev Immunol 2001, 21, 249-261).

The radio-immunoconjugate of the anti-PSMA monoclonal antibody (mAb) 7E11, known as the PROSTASCINT® scan, is currently being used to diagnose prostate cancer metastasis and recurrence. However, this agent tends to produce images that are challenging to interpret (Lange, P. H. PROSTASCINT scan for staging prostate cancer. Urology 2001, 57, 402-406; Haseman, M. K.; et al. Cancer Biother Radiopharm 2000, 15, 131-140; Rosenthal, S. A.; et al. Tech Urol 2001, 7, 27-37). More recently, monoclonal antibodies have been developed that bind to the extracellular domain of PSMA and have been radiolabeled and shown to accumulate in PSMA-positive prostate tumor models in animals. However, diagnosis and tumor detection using monoclonal antibodies has been limited by the low permeability of the monoclonal antibody in solid tumors.

The selective targeting of cancer cells with radiopharmaceuticals, either for imaging or therapeutic purposes is challenging. A variety of radionuclides are known to be useful for radio-imaging or cancer radiotherapy, including ¹¹¹In, ⁹⁰Y, ⁶⁸Ga, ¹⁷⁷Lu, ^{99m}Tc, ¹²³I and ¹³¹I. Recently it has been shown that some compounds containing a glutamate-urea-glutamate (GUG) or a glutamate-urea-lysine (GUL) recognition element linked to a radionuclide-ligand conjugate exhibit high affinity for PSMA.

In WO 2015/055318 new imaging agents with improved tumor targeting properties and pharmacokinetics were described. These compounds comprise a motif specifically binding to cell membranes of cancerous cells, wherein said motif comprises a prostate-specific membrane antigen (PSMA), that is the above mentioned glutamate-urea-lysine motif. The preferred molecules described in WO 2015/055318 further comprise a linker which binds via an amide bond to a carboxylic acid group of DOTA as chelator. Some of these compounds have been shown to be promising agents for the specific targeting of prostate tumors. The compounds were labeled with ¹⁷⁷Lu (for therapy purposes) or ⁶⁸Ga (for diagnostic purposes) and allow for visualization and targeting of prostate cancer for radiotherapy purposes.

However, in therapeutic applications of radioactively labeled PSMA inhibitors, organs with physiological PSMA expression turned out to be dose limiting and thus minimize the therapeutic success. In particular, the high renal and salivary gland uptake of the radioactively labeled PSMA inhibitor substances is noticeable, which, in the case of a therapeutic application, gives rise to considerable side effects. Attempts to improve the kidney uptake of PSMA inhibitors has led to the development of PSMA-617 [Benesova, M., et al. (2016) J Med Chem 59, 1761-75], a compound which is already used clinically with 177Lu or 225Ac for endoradiotherapy of prostate cancer. However, a reduction in salivary and lacrimal gland uptake has not yet been achieved and is still described as critical and dose-limiting in early clinical work. In a first-in-man study with 225Ac-PSMA-617, two patients with extremely advanced and end-stage disease showed complete remission. In both patients the PSA value fell below the detectability limit. Accompanying diagnostic recordings with 68Ga-PSMA-11 confirmed a complete response.

As already mentioned above, the strong accumulation of PSMA ligands in non-target tissues, in particular the salivary and lacrimal glands, which has been described in numerous scientific publications leads to considerable side effects. The salivary and lacrimal glands may be severely and partially irreversibly damaged, in particular during alpha therapy with 225Ac. The resulting xerostomia for example represents a dose-limiting side effect. To resolve this issue, improvement of tissue specificity of PSMA ligands was proposed e.g. in WO 2020/165420 A1.

Thus, there is still the need for improved PSMA ligands which provide advantageous options for the detection, treatment and management of PSMA-expressing cancers, in particular prostate cancer. The technical problem underlying the present invention can be seen as the provision of PSMA ligands and methods for complying with the aforementioned needs. The technical problem is solved by the embodiments characterized in the claims and herein below.

### SUMMARY OF THE INVENTION

The solution of said objective is achieved by providing the embodiments characterized in the claims. The inventors found new compounds which are dye labelled, preferably fluorescent dye labelled, complexing PSMA ligands. In accordance, the compounds may be used e.g. in intraoperative or diagnostic labelling of PSMA-expressing cells, but also in nuclear medicine as tracers, imaging agents and for the treatment of various disease states of PSMA-expressing cancers, in particular prostate cancer. Thus, the PSMA binding ligands described herein can e.g. be used in preoperative PET Imaging and in Fluorescence-Guided Surgery of cancers, especially prostate cancer, and metastases thereof. These compounds are described in more detail below.

In particular, the present invention relates to a PSMA binding ligand or a pharmaceutically acceptable salt or solvate thereof comprising a PSMA binding motif Q, a chelator residue A, a dye group Z and at least one linker L^{BQ} comprising at least one amino acid X₁, preferably wherein X₁ is a N— alkylated amino acid, more preferably an N-methylated amino acid, in particular wherein X¹ is —N(CH₃)—CH₂—C(═O)—.

Further, the present invention relates to a complex comprising
(a) a radionuclide, and
(b) the PSMVA binding ligand as described above and below or a pharmaceutically acceptable salt or solvate thereof.

Further, the present invention relates to a pharmaceutical composition comprising a PSMA binding ligand, as described above or below, or a pharmaceutically acceptable salt or solvate thereof, as described above or below, or a complex, as described above or below.

Further, the present invention relates to a PSMA binding ligand, as described above or below, or a pharmaceutically acceptable salt or solvate thereof, or a complex, as described above or below, or a pharmaceutical composition as described above or below, for use in treating or preventing PSMA-expressing cancers, in particular prostate cancer, and/or metastases thereof.

Further, the present invention relates to PSMA binding ligand and/or a complex as described above or below as a labeling agent for detecting cancerous tissue in a subject.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s).

The use of the PSMA ligand and/or the complex labeling compound, as referred to herein, comprises at least administration of a labeling dose of said labeling compound. The use may, however, in addition comprise further steps before, concomitant to, and/or after said administration deemed appropriate by the skilled person. The use, preferably, additionally comprises at least one step as specified herein, in particular a step of a use and/or a step of a method as described herein. Preferably, the use comprises intraoperative identification of cancerous tissue.

As described above, the PSMA binding ligand comprises a PSMA binding motif Q, a chelator residue A, a dye group Z and at least one linker L^{BQ}.

Preferably, the PSMA binding ligand has the structure

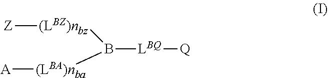

wherein Q is the PSMA binding motif,
A is the chelator residue,
Z is the dye group,
B is a branching group,
L^{BQ} is a linker connecting Q with B, said linker comprising the at least one amino acid X₁,
L^{BZ} is a linker, wherein n_{bz} is 0 or 1
and L^{BA} is a linker, wherein n_{ba} is 0 or 1.

### PSMA Binding Motif Q

The PSMA binding motif Q has preferably the structure

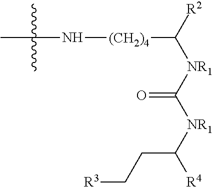

wherein R¹ is H or —CH₃, preferably H, wherein R², R³ and R⁴ are independently of each other, selected from the group consisting of—CO₂H, —SO₂H, —SO₃H, —OSO₃H, —PO₂H, —PO₃H and —OPO₃H₂. More preferably, R², R³ and R⁴ are CO₂H. In particular, R¹ is H and R², R³ and R⁴ are CO₂H. The wavy line indicates the connection site to the linker L^{BQ}.

### Chelator Residue A

A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid](NOPO), 3,6,9,15-tetraazabicyclo[9.3.1]pentadeca-1 (15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA)

The term “a chelator residue” and typically also the term “chelator residue derived from a chelator selected from the group” is denoted to mean that the above mentioned chelators, thus typically the chelators defined in the “group”, have been linked, via a suitable functional group, to the rest of the PSMA binding ligand, preferably to the linker L^{BA}.

More preferably chelators defined in the “group”, have been linked, via a former carboxylic acid group of the chelator, to an NH group of the rest of the PSMA molecule, such as to an amine group L^{BA}, thereby forming an amide bond between the chelator and the rest of the pSMA binding ligand, such as with and L^{BA}.

Preferably, A is a chelator residue having a structure selected from the group consisting of

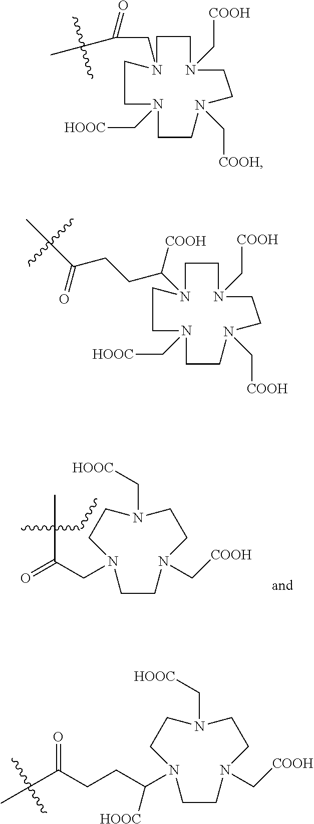

Most preferably, A has the structure

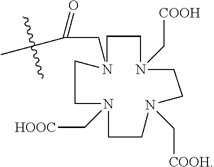 [SMILES: *C(=O)CN1CCN(CC(=O)O)CCN(CC(=O)O)CCN(CC(=O)O)CC1]

The at least one linker L^{BQ}.

As described above, the PSMA ligand preferably comprises at least one linker L^{BQ} comprising at least one amino acid X₁.

It is to be understood that the term amino acid includes all naturally-occurring and non-naturally-occurring amino acids, including all stereoisomers, such as enantiomers and diastereomers of these amino acids. Most preferably, the amino acids are alpha amino acids. With respect to the chirality, L-amino acids are preferred.

Preferably X₁ is
a neutral amino acid, or
a N-alkylated amino acid, preferably N-methylated amino acid,

More preferably X₁ is a N-alkylated amino acid, more preferably N-methylated amino acid, such as a neutral N-alkylated amino acid, more preferably a neutral N-methylated amino acid.

The term “neutral amino acid” as used within the meaning of the present invention includes each and every amino acid having no net charge at a pH of 7. It is to be understood that the term includes all naturally-occurring and non-naturally-occurring amino acids, including all stereoisomers, such as enantiomers and diastereomers of these amino acids, such glycine, alanine, valine, isoleucine, phenylalanine, beta-alanine as well as unnatural amino acids comprising a neutral linker between N and C terminus, such at least one —(CH2-CH2-O)— group between the N-terminus and the C-terminus, as well as N-alkylated such as N-methylated amino acids, thus amino acids comprising instead of a proton —H an alkyl or Methyl group attached to the amino group of the amino acid.

Preferably, the amino acid X₁ is an N-alkylated amino acid. This term refers to amino acids which comprise instead of a proton —H an alkyl, such as a methyl, attached to the amino group of the amino acid. N-alkyl amino acids are also referred to hereunder as N-alkylated or alkylated amino is acid. N-methyl amino acids are also referred to hereunder as N-methylated or methylated amino acid.

Preferred N-alkylated amino acids, include, but are not limited to, N-alkyl-glycine, N-alkyl-alanine, N-alkyl-valine, N-alkyl-isoleucine, N-alkyl-leucine, N-alkyl-methionine, N-alkyl-phenylalanine, N-alkyl-tyrosine or N-alkyl-tryptophane. Preferably X1 is a neutral N-alkylated amino acid.

More preferably, the amino acid X₁ is an N-methylated amino acid, preferably a neutral N-methylated amino acid.

It is to be understood that also this term includes N-methyl derivatives of all naturally-occurring and non-naturally-occurring amino acids, including all stereoisomers, such as enantiomers and diastereomers of these amino acids. Most preferably, the amino acids are N-methyl alpha amino acids. With respect to the chirality, L-amino acids are preferred.

The term methylated amino acid or N-methyl amino acid includes, but is not limited to, N-methyl arginine, N-methyl histidine, N-methyl lysine, N-methyl aspartic acid, N-methyl glutamic acid, N-methyl serine, N-methyl threonine, N-methyl asparagine, N-methyl glutamine, N-methyl cysteine, selenocysteine, N-methyl glycine, N-methyl proline, N-methyl alanine, N-methyl valine, N-methyl isoleucine, N-methyl leucine, N-methyl methionine, N-methyl phenylalanine, N-methyl tyrosine, N-methyl tryptophane.

More preferably, the at least one N-methyl amino acid is a neutral amino acid, such as N-methyl glycine, N-methyl alanine, N-methyl valine, N-methyl isoleucine, N-methyl leucine, N-methyl methionine, N-methyl phenylalanine, N-methyl tyrosine or N-methyl tryptophane,

More preferably the at least one N-methyl amino acid is selected from the group consisting of N-methyl glycine, N-methyl alanine, N-methyl valine, N-methyl isoleucine, N-methyl leucine, N-methyl phenylalanine,

More preferably, the methylated amino acid is N-methyl alanine or N-methyl glycine (sarkosine), more preferably N-methyl glycine, thus the at least one amino acid preferably has the structure —N(CH₃)—CH₂—C(═O)— or —N(CH₃)—CH(CH₃)—C(═O)—, more preferably —N(CH₃)—CH₂—C(═O)—.

Preferably, the linker L^{BQ} comprises an amino acid sequence AA comprising the building block (X₁)_{b1}, wherein n1 is in the range of from 2 to 25. In case, the linker comprises more than one amino acid, the amino acids may be the same or may be different from each other. Preferably, the amino acid sequence AA consist of the block (X₁)ₙ₁, wherein all amino acids X₁ are the same. Thus, the linker may e.g. comprise —N(CH₃)—CH₂—C(═O)— and —N(CH₃)—CH(CH₃)—C(═O)— groups, or only —N(CH₃)—CH₂—C(═O)— or —N(CH₃)—CH(CH₃)—C(═O)— groups or only —N(CH₃)—CH₂—C(═O)— groups.

Preferably, the linker comprises at least 3, preferably 3 to 25 methylated amino acids, which may be the same or different.

According to a preferred embodiment, at least two N-methylated amino acids are attached to each other.

Preferably, the linker comprises the building block —(X1)n1, wherein X¹ is preferably a N-alkylated, preferably a methylated amino acid, more preferably a neutral methylated amino acid, more preferably wherein X¹ is —N(CH₃)—CH₂—C(═O)—, and wherein n1 is an integer of from 1 to 25, mote preferably 3 to 20, more preferably 5 to 15, such as 5, 6, 7, 8, 9, 10, 11, 12, 23, 24, 15, more preferably 5 or 10, in particular 10.

Preferably, the PSMVA binding ligand, in particular linker L^{BQ}, further comprises in addition to X₁ or (X₁)ₙ₁, at least one amino acid building block AS^{a} and/or at least one amino acid building block AS^{b}.

### Amino Acid Building Block AS^{a}

The amino acid building block AS^{a} preferably has the structure

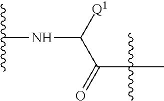

wherein Q¹ is selected from the group consisting of alkylaryl, arylalkyl, aryl, alkylheteroaryl, heteroarylalkyl and heteroaryl.

The term “aryl”, as used in this context of the invention, means optionally substituted, 5- and 6-membered aromatic rings, and substituted or unsubstituted polycyclic aromatic groups (aryl groups), for example tricyclic or bicyclic aryl groups. Optionally substituted phenyl groups or naphthyl groups may be mentioned as examples. Polycyclic aromatic groups can also contain non-aromatic rings.

The term “alkylaryl” as used in this context of the invention refers to aryl groups in which at least one proton has been replaced with an alkyl group (Alkyl-aryl-).

The term “arylalkyl” as used in this context of the invention refers to aryl groups linked via an alkyl group (Aryl-alkyl-).

The term “heteroaryl”, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to heteroaryl groups in which at least one proton has been replaced with an alkyl group (Alkyl-Heteroaryl-).

The term “heteroarylalkyl” as used in this context of the invention refers to heteroaryl groups linked via an alkyl group (Heteroaryl-alkyl-).

The term “cycloalkyl” means, in the context of the invention, optionally substituted, cyclic alkyl residues, wherein they can be monocyclic or polycyclic groups. Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as 0, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q1 comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably Q¹ is selected from the group consisting of:

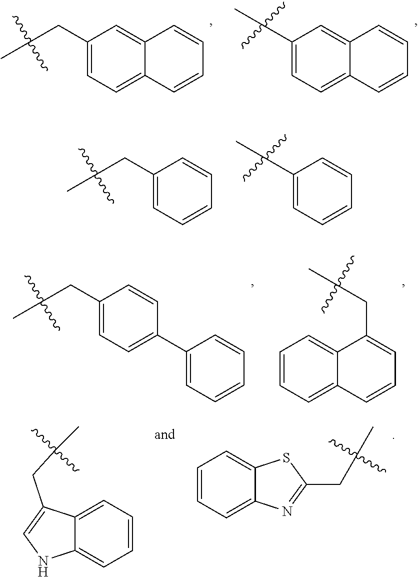

wherein Q¹ is most preferably

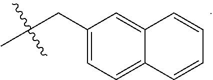 [SMILES: *Cc1ccc2ccccc2c1]

### Amino Acid Building Block AS^{b}

The amino acid building block AS^{b} preferably has the structure (b)

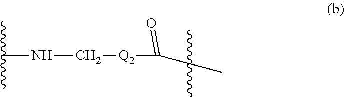

wherein Q² is selected from the group consisting of aryl, alkylaryl, arylalkyl, cycloalkyl, heterocycloalkyl, heteroaryl, heteroarylalkyl and alkylheteroaryl.

The term “aryl”, as used in this context of the invention refers to optionally substituted, 5- and 6-membered aromatic rings, and substituted or unsubstituted polycyclic aromatic groups (aryl groups), for example tricyclic or bicyclic aryl groups (—Ar—). Optionally substituted phenyl groups or naphthyl groups may be mentioned as examples. Polycyclic aromatic groups can also contain non-aromatic rings, the Aryl group in this context of the invention

The term “alkylaryl” as used in this context of the invention refers to aryl groups in which at least one proton has been replaced with an alkyl group (-alkyl-aryl-) and which are linked via to alkyl group to the —CH2- group and via the aryl group to the carbonyl group.

The term “arylalkyl” as used in this context of the invention refers to aryl groups linked via an alkyl group to the carbonyl group and via the aryl group to the —CH2- group (-aryl-alkyl-).

The term “heteroaryl” (-Heteraryl-, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to aryl groups in which at least one proton has been replaced with an alkyl group (-alkyl-heteroaryl-) and which are linked via to alkyl group to the —CH2- group and via the heteroaryl group to the carbonyl group.

The term “heteroarylalkyl” as used in this context of the invention refers to heteroaryl groups linked via an alkyl group to the carbonyl group and via the heteroaryl group to the —CH2- group (-aryl-alkyl-).

The term “cycloalkyl” (-cycloalkyl-) means, in the context of the invention, optionally substituted, cyclic alkyl residues, wherein they can be monocyclic or polycyclic groups. Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q² is an aryl group or cycloalkylgroup, more preferably

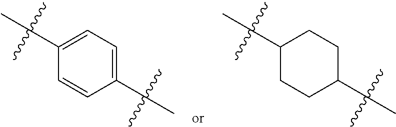 [SMILES: *c1ccc(*)cc1 ; *C1CCC(*)CC1]

most preferably

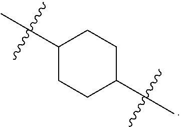 [SMILES: *C1CCC(*)CC1]

It is to be understood that any stereoisomers of Q² are possibly and included. In case Q² is

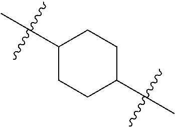 [SMILES: *C1CCC(*)CC1]

it is to be understood that this includes the cis as well as the trans isomer, with the trans isomer being particularly preferred.

Preferably, the PSMVA binding ligand, preferably the linker L^{BQ}, described above and below, comprises at least one amino acid building block AS^{a} and at least one amino acid building block AS^{b}, wherein AS^{a} has the structure

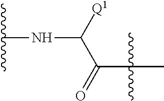

wherein Q¹ is selected from the group consisting of alkylaryl, arylalkyl, aryl, alkylheteroaryl, heteroarylalkyl and heteroaryl, and wherein AS^{b} has the structure (b)

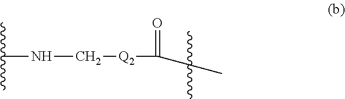

wherein Q² is selected from the group consisting of aryl, alkylaryl, arylalkyl, cycloalkyl, heterocycloalkyl, heteroaryl, heteroarylalkyl and alkylheteroaryl, preferably wherein Q² is

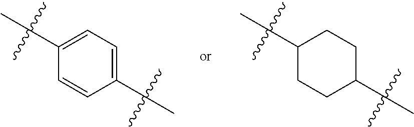 [SMILES: *c1ccc(*)cc1 ; *C1CCC(*)CC1]

more preferably

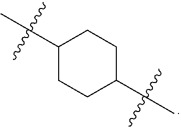 [SMILES: *C1CCC(*)CC1]

preferably wherein the PSMA binding ligand comprises the linking unit -(AS^{b})_{q}AS^{a}-, wherein q is an integer of from 0-3, preferably wherein q is 1.

Preferably, the PSMA binding motif Q is linked to the group -(AS^{b})_{q}, more preferably to the moiety -(AS^{b})_{q}-AS^{a}. Thus, the PSMA binding ligand preferably comprises the building block

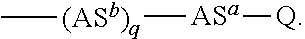

More preferably, the building block -L^{BQ}-Q comprises, preferably consists of, the building block —(X¹)ₙ₁-(AS^{b})_{q}-AS^{a}-Q in particular having the structure 1.

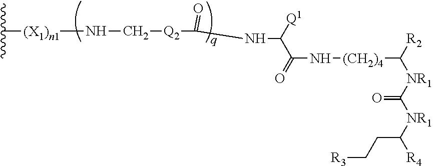

wherein R¹ is H or —CH₃, preferably H, wherein R², R³ and R⁴ are independently of each other, selected from the group consisting of—CO₂H, —SO₂H, —SO₃H, —OSO₃H, —PO₂H, —PO₃H and —OPO₃H₂,
Q¹ is selected from the group consisting of alkylaryl, arylalkyl, aryl, alkylheteroaryl, heteroarylalkyl and heteroaryl,
Q² is selected from the group consisting of aryl, alkylaryl, arylalkyl, cycloalkyl, heterocycloalkyl, heteroaryl, heteroarylalkyl and alkylheteroaryl,
and wherein q is an integer of from 0-3,
wherein X¹ is as described above, preferably wherein X¹ is a methylated amino acid, more preferably a neutral N-methyl amino acid, wherein X¹ is more preferably wherein X¹ is —N(CH₃)—CH₂—C(═O)—, and wherein n1 is an integer of from 1 to 25, mote preferably 3 to 20, more preferably 5 to 15, such as 5, 6, 7, 8, 9, 10, 11, 12, 23, 24, 15, more preferably 5 or 10, in particular 10.

### The Branching Group B

As describe above, the present invention preferably relates to a PSMA binding ligand having the structure

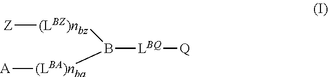

wherein B is a branching group. The term “branching group” as used herein refers to a group that divides a molecular chain into at least two molecular chains. Preferably, the branching group is an amino acid, having a side chain with a suitable functional group to which the group -(L^{BZ})n_{bz}-Z or -(L^{BA})n_{ba}-A can be attached. Preferably, the group B has the structure (B1)

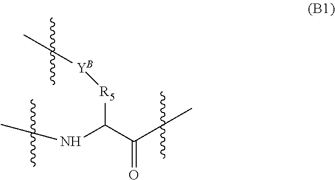

wherein R₅ is an alkyl group, preferably a —(CH₂)₁₋₁₀ group, more preferably a —(CH₂)₂₋₄ group, and Y^{B} is a functional group linking R₅ to the group -(L^{BZ})n_{bz}-Z or -(L^{BA})n_{ba}-A, wherein Y^{B} is preferably a group —NH—, —O—, —S—, —NH—NH—, —NH—NH—C(═O)—, —C(═O)—NH—NH—or C(═O)—, more preferably NH— or —C(═O)—. Most preferably R₅ is (CH₂)₄ and Y^{B} is —NH—.

Preferably, the PSMA binding ligand thus has one of the following structures (Ia) or (Ib), preferably the structure (Ia)

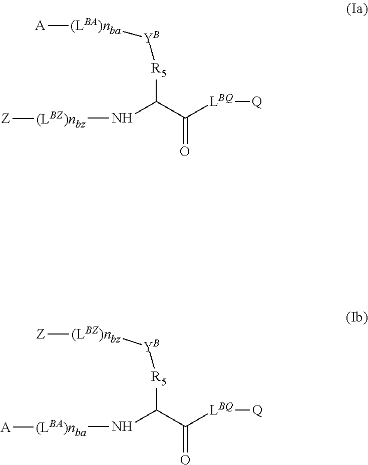

wherein R₅ is an alkyl group, preferably a —(CH₂)₁₋₁₀ group, more preferably a —(CH₂)₂₋₄ group, and Y^{B} is a functional group linking R₅ to the group -(L^{BZ})n_{bz}-Z or -(L^{BA})n_{ba}-A, wherein Y^{B} is preferably a group —NH—, —O—, —S—, —NH—NH—, —NH—NH—C(═O)—, —C(═O)—NH—NH—or C(═O)—, more preferably NH— or —C(═O)—. Most preferably R₅ is (CH₂)₄ and Y^{B} is —NH—.

More preferably, the PSMA ligand has the structure (IIa) or (JIb)

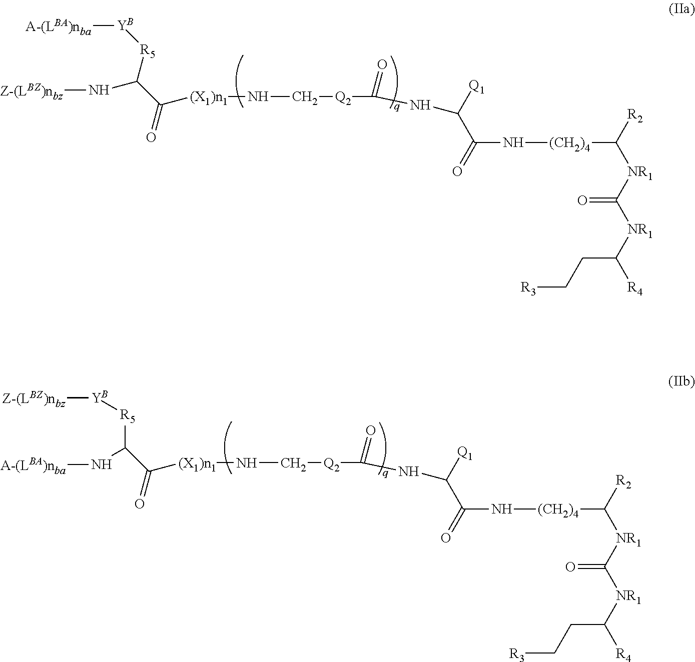

wherein R¹ is H or —CH₃, preferably H, wherein R², R³ and R⁴ are independently of each other, selected from the group consisting of—CO₂H, —SO₂H, —SO₃H, —OSO₃H, —PO₂H, —PO₃H and —OPO₃H₂, Q¹ is selected from the group consisting of alkylaryl, arylalkyl, aryl, alkylheteroaryl, heteroarylalkyl and heteroaryl, Q² is selected from the group consisting of aryl, alkylaryl, arylalkyl, cycloalkyl, heterocycloalkyl, heteroaryl, heteroarylalkyl and alkylheteroaryl, and wherein q is an integer of from 0-3, wherein X¹ is, as described above, preferably a methylated amino acid, more preferably a neutral methylated amino acid, more preferably wherein X¹ is —N(CH₃)—CH₂—C(═O)— and n1 is an integer of from 1 to 25, wherein
Z is the dye group,
L^{BZ} is a linker, wherein n_{bz} is 0 or 1
and L^{BA} is a linker, wherein n_{ba} is 0 or 1, and wherein R₅ is an alkyl group, preferably a —(CH₂)₂₋₄ group, and Y^{B} is a functional group linking R₅ to the group -(L^{BA})n_{ba}-A or to the group -(L^{BZ})n_{bz}-Z, wherein Y^{B} is preferably a group —NH— or —C(═O)—, more preferably a —NH— group.

Thus, the PSMVA ligand preferably has the structure (IIIa) or (IIIb)

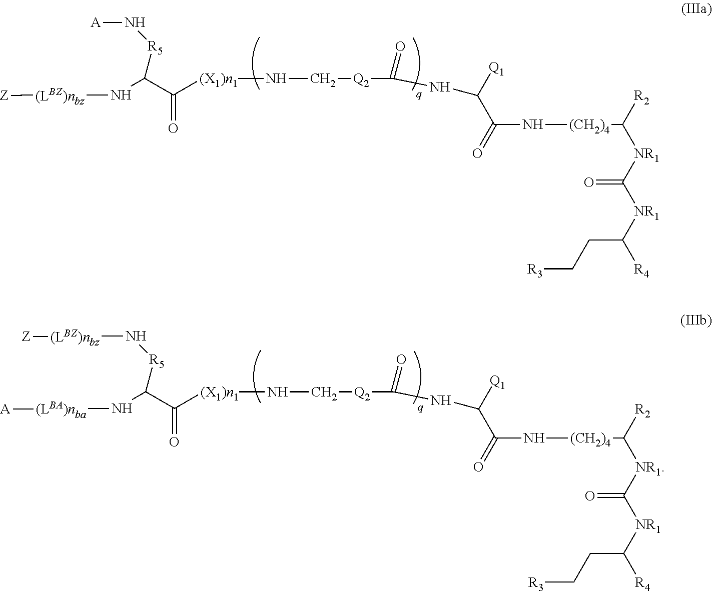

more preferably the structure (IVa) or (IVb)

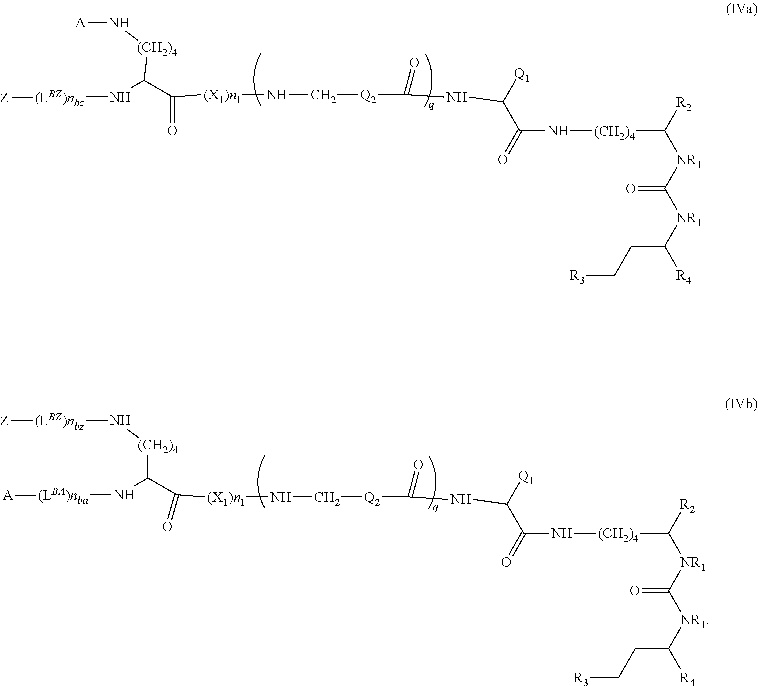

The Linker L^{BQ} and or L^{Bz}

Any linker suitable for attaching Z and A respectively to the branching group B, preferably to the amino groups of the structure (B1), may be used. Preferably, the linker L^{BQ} and/or L^{BZ} comprise at least one amino acid. This includes all naturally-occurring and non-naturally-occurring amino acids, including all stereoisomers, such as enantiomers and diastereomers of these amino acids. Most preferably, the amino acids are alpha amino acids. With respect to the chirality, L-amino acids are preferred. Preferably the linker L^{BQ} and/or L^{BZ} comprise at least one neutral amino acid or at least one methylated amino acid, preferably at least one neutral N-methyl amino acid, more preferably —N(CH₃)—CH₂—C(═O)—.

According to a first preferred embodiment, the PSMA ligand has the structure (Ia), preferably (IIa), more preferably (IIIa), more preferably (IVa). In this case, L^{BZ} preferably comprises an amino acid sequence, said sequence preferably comprising the at least one neutral amino acid or at least one methylated amino acid, preferably at least one neutral N-methyl amino acid, more preferably —N(CH₃)—CH₂—C(═O)—.

Preferably, Z is attached to the N-terminal end of L^{BZ} or to a functional group of a side chain of an amino acid present in L^{BZ}, with the proviso that in case L^{BZ} is attached to a side chain of an amino acid present in L^{BZ}, the N-terminus of the amino acid chain is preferably acylated with a group —C(═O)—R^{z}, with R^{z} being preferably an C10-C20-alkyl chain.

According to one preferred embodiment, Z is attached to the N-terminal end of an amino acid chain. In this case, L^{BZ} preferably comprises, more preferably consist of, an amino acid sequence having structure —(X^{2bz})_{n2bz}—(X^{1bz})_{n1bz}— and Z is attached to the N-terminal end of —(X^{2bz})_{n2bz} —(X^{1bz})_{n1bz}, herein X^{1bz} is preferably a methylated amino acid, more preferably wherein X^{1bz} is —N(CH₃)—CH₂—C(═O)— and n1bz is of from 1 to 20, preferably 4 to 10, more preferably 5, and wherein X^{2bz} is preferably a neutral amino acid, more preferably beta-Ala and n2bz is of from 1 to 20, preferably 1. In this case, the PSMA ligand preferably has the structure

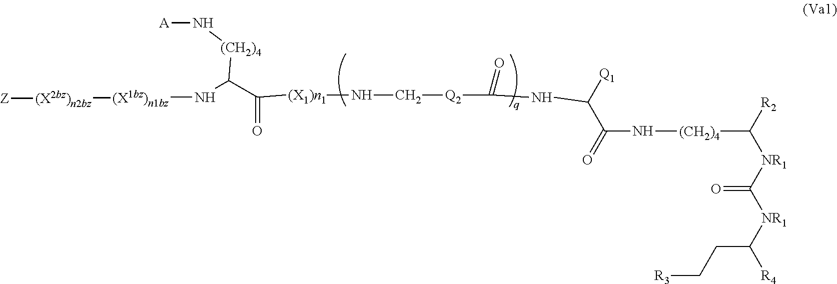

According to a alternative embodiment, Z is attached to the side chain of an amino acid present in the amino acid chain. In this case, the amino acid sequence preferably has structure —(X^{3bz})_{n3bz}—(X^{1bz})_{nb1z}— and wherein X^{1bz} is preferably a methylated amino acid, more preferably wherein X^{1z} is —N(CH₃)—CH₂—C(═O)— and n1bz is of from 1 to 20, preferably 4 to 10, more preferably 5, and wherein X^{2bz} is glutamic acid or lysine, preferably lysine and Z is attached to the side chain of X^{2bz}, and n2z is of from 1 to 20, preferably 1. In this case, the N terminus is preferably acylated. Preferably, the N-terminus is acylated with a group —C(═O)—R^{z}, with R^{z} being preferably an C10-C20-alkyl chain. In this case, the PSMA ligand preferably has the structure (Va2), more preferably (VIa2)

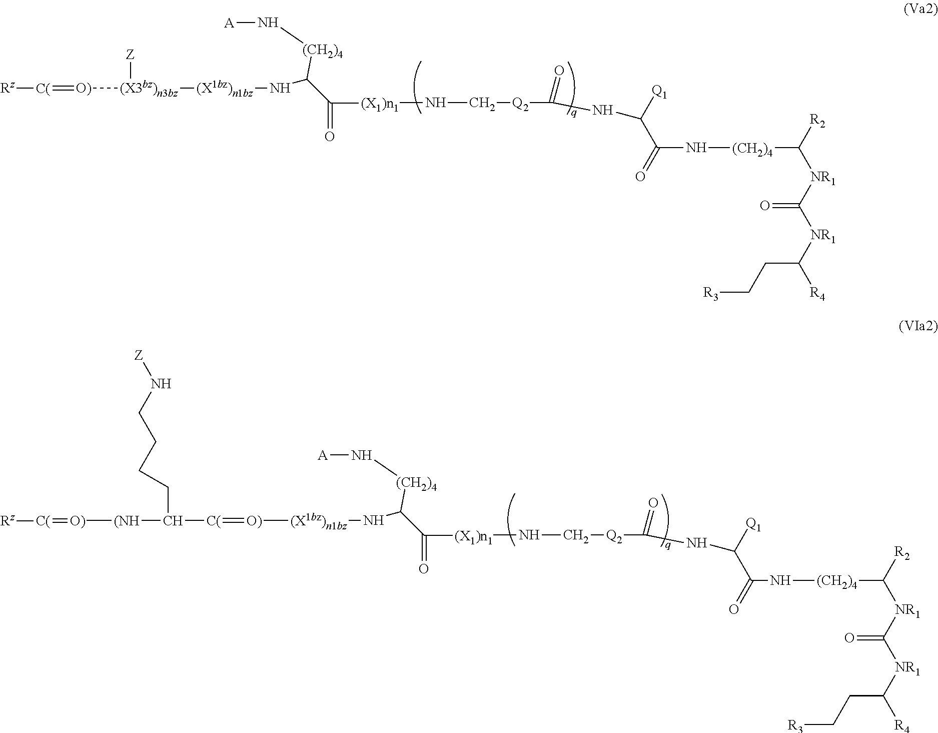

According to a second preferred embodiment, the PSMA ligand has the structure (Ib), preferably (IIb), more preferably (IIIb), more preferably (IVb). In this case, L^{BA} preferably comprises an amino acid sequence, said sequence preferably comprising at least on methylated amino acid, preferably —N(CH₃)—CH₂—C(═O)—. Preferably, L^{BA} comprises an amino acid sequence, and A is attached to the N-terminal end of L^{BA} or to a functional group of a side chain of an amino acid present in L^{BA}, with the proviso that in case L^{BA} is attached to a side chain, the N-terminus is preferably acylated with a group —C(═O)—R^{z}, with R^{z} being preferably an C10-C20-alkyl chain. More preferably, L^{BA} comprises, preferably consists of, the amino acid sequence —(X^{2a})ₙ₂ₐ—(X^{1ba})_{n1ba}— and A is attached to the N-terminal end of —(X^{2ba})_{n2ba}—(X^{1ba})_{n1ba}—, and wherein X^{1ba} is a preferably a methylated amino acid, more preferably wherein X^{1ba} is —N(CH₃)—CH₂—C(═O)— and n1a is of from 1 to 20, preferably 4 to 10, more preferably 5, and wherein X^{2ba} is a neutral amino acid, preferably beta-Ala and n2a is of from 1 to 20, preferably 1.

### The Dye Group Z

As used in the context of the present invention, the terms “dye group”, “dye moiety”, “label” and “stain” may be understood interchangeably in the broadest sense as any moiety with the above formula that provides a visible stain. Preferably, the dye moiety is a fluorescent dye moiety and/or a chromatic moiety, particularly preferably the dye is a fluorescent dye.

A fluorescent dye as used herein may be understood in the broadest sense as any dye moiety enabling fluorescence detection. Preferably, such fluorescence detection is in a range of from 350 to 1000 nm, i.e. in the visible spectrum and in the Near Infrared (NIR) spectrum, in particular in a range of from 400 to 850 nm, i.e. in the visible spectrum. Preferably, the fluorescence signal emitted by the fluorescence dye moiety is well-distinguishable from the autofluorescence of cancer and surrounding tissue. Numerous fluorescent dye moieties are known in the art, and will be readily apparent to one of ordinary skill. Many fluorescent dyes are commercially available with activated groups used to react with protein sidechains or other compounds such as precursor compounds for the preparation of a compound of the present invention. Preferably, the dye enables a fluorescence detection by radiation with a wavelength in a range of from with a wavelength of from 350 to 1000 nm, i.e. in the visible spectrum and in the Near Infrared (NIR) spectrum, preferably of from 400 to 850 nm, such as 600 nm to 850, preferably 700 nm to 780 nm and/or allows for a detection of fluorescence by eye and/or at a wavelength in the range of from 780 nm to 850 nm.

Additionally or alternatively, the dye may also be chromatic, i.e., provoke a colour perception when illuminated by any light. Such chromatic effect may be provoked by absorbing light of one or more particular wavelength range(s) in the visible range (i.e., in range(s) from approximately 350 nm to approximately 800 nm) and/or by emitting light of one or more particular wavelength range(s) in the visible range. Preferably, the colour is different from the neoplasia and the surrounding tissue intended to be examined. Therefore, a dye moiety, when not intended for fluorescence detection, is preferably not red or brown, but rather preferably blue or green. When the dye is intended for fluorescence detection, the difference in colour will typically play a minor role as long as the fluorescence is detectable over the autofluorescence background. Preferably, the chromatic dye moiety in the context of the present invention is a small-molecule dye, i.e., a dye moiety having a molecular weight (MW) of not more than 1000 Da, preferably not more than 750 Da, in particular nor more than 500 Da.

Depending on the chemical properties of the fluorescence dye in the compound of the present invention and the presence of fluorophore(s) and/or quenchers on the surface of the target cells, i.e., the cell membranes of the respective cancer cells, also enable to observe effects such as fluorescence energy transfer (FRET) and/or fluorescence quenching upon binding of the compound according to the present invention to said cell membranes. Additionally or alternatively, the presence of the fluorescence dye ties also enables to conduct further examination methods based on fluorescence such as, e.g., fluorescence recovery after photobleaching (FRAP), fluorescence loss in photobleaching (FLIP). These methods may provide information on the mobility of the compound or salt thereof bound to or associated with the cell membranes of a cancer cell.

Preferably, none of the chemical groups present in the PSMA ligand nor the complexed radiometal quench the intensity of the fluorescence signal obtainable from the dye Z at its emission maximum in an aqueous environment of approximately neutral pH (i.e., pH 6-8, in particular 6.5-7.5) by more than 50%.

Preferably, the dye Z is suitable for emit light in an aqueous environment of approximately neutral pH, i.e., pH 6-8, in particular 6.5-7.5, in particular pH 7.0-7.5.

In a preferred embodiment, the dye Z is a fluorescent dye having an emission maximum in the range from 350 nm to 1000 nm, preferably 400 nm to 850 nm.

According to is selected from the group consisting of AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750, Cy2, Cy3, Cy3B, Cy3.5, Cy5, sulfoCy5, Cy5.5 and Cy7, DyLight 350, DyLight 405, DyLight 488, DyLight 550, DyLight 594, DyLight 633, DyLight 650, DyLight 680, DyLight 750 and DyLight 800, FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid), 7-Hydroxy-4-methylcoumarin-3, 7-Hydroxycoumarin-3-carboxylic acid (Pubchem SID 135727263), MCA (7-Methoxycoumarin-4-acetic acid) (Pubchem CID 342221), 7-Methoxycoumarin-3, AMF (4′-(Aminomethyl)fluorescein), 5-DTAF (5-(4,6-Dichlorotriazinyl)aminofluorescein), 6-DTAF (6-(4,6-Dichlorotriazinyl)aminofluorescein), 6-FAM (6-Carboxyfluorescein), 5 (6)-FAM cadaverine (Fluorescein-5 (6)-carboxamide cadaverine), 5-FAM cadaverine (Fluorescein-5-carboxamide cadaverine), 5 (6)-FAM ethylenediamine (Fluorescein-5 (6)-carboxamide ethylenediamine), 5-FAM ethylenediamme (Fluorescein-5 (6)-carboxamide ethylenediamine), 5-FITC (FITC Isomer I; fluorescein-5-isothiocyanate), 5-FITC cadaverin; Fluorescein-5-maleimide; 5-IAF (5-Iodoacetamidofluorescein), 6-JOE (6-Carboxy-4′,5′-dichloro-2′,7′-dimethoxyfluorescein), 5-CR110 (5-Carboxyrhodamine 110), 6-CR110 (6-Carboxyrhodamine 110), 5-CR6G (5-Carboxyrhodamine 6G); 6-CR6G (6-Carboxyrhodamine 6G), 5 (6)-Caroxyrhodamine 6G cadaverine; 5 (6)-Caroxyrhodamine 6G ethylenediamme, 5-ROX (5-Carboxy-X-rhodamine), 6-ROX (6-Carboxy-X-rhodamine); 5-TAMRA (5-Carboxytetramethylrhodamine), 6-TAMRA (6-Carboxytetramethylrhodamine), 5-TAMRA cadaverine, 6-TAMRA cadaverine, 5-TAMRA ethylenediamine (5-Carboxytetramethylrhodamine ethylenediamine), 6-TAMRA ethylenediamine (6-Carboxytetramethylrhodamine ethylenediamine), 5-TMR C6 maleimide, 6-TMR C6 maleimide, TR C2 maleimide, TR cadaverine, 5-TRITC (Tetramethylrhodamine-5-(and-6)-isothiocyanate), 6-TRITC, R isomer (Tetramethylrhodamine-6-isothiocyanate), Dansyl cadaverine (5-Dimethylaminonaphthalene-1-(N-(5-aminopentyl))sulfonamide), EDANS C2 maleimide (5-((2-Aminoethyl)amino)naphthalene-1-sulfonic acid C2 maleimide), EDANS acid (5-((2-Aminoethyl)amino)naphthalene-1-sulfonic acid); fluorescamine (4-Phenylspiro-[furan-2 (3H),1-phthalan]-3,3′-dion), NBD (4-Chlor-7-nitro-benzo-2-oxa-1,3-diazol), pyrromethene, Texas Red (1H,5H,11H,15H-Xantheno[2,3,4-j:5,6,7-i′j′]diquinolizin-18-ium, 9-[2(or 4)-(chlorosulfonyl)-4(or 2)-sulfophenyl]-2,3,6,7,12,13,16,17-octahydro-, inner salt), Cy5, Cy5 succinimidylester (3H-Indolium, 2-[5-[1-[6-[(2,5-dioxo-1-pyrrolidinyl)oxy]-6-oxohexyl]-1,3-dihydro-3,3-dimethyl-5-sulfo-2H-indol-2-ylidene]-1,3-pentadien-1-yl]-1-ethyl-3,3-dimethyl-5-sulfo-, inner salt), Cy5 sacridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, 2-aminonaphthalen, indicyano green (ICG), IRDye800CW, Bodipy650-X, CF680R, 580CP-methocy, 610CP, SiR-methyl, ATTO 390, ATTO 425, ATTO 465, ATTO 488, ATTO 495, ATTO 514, ATTO 520, ATTO 532, ATTO Rho6G, ATTO 550, ATTO 565, ATTO Rho3B, ATTO Rho11, ATTO Rho12, ATTO Thio12, ATTO Rho101, ATTO 590, ATTO Rho13, ATTO 594, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO647N, STAR 600, STAR635 P, STAR RED, 580CP-methoxy and derivatives thereof.

Preferably, Z is selected from the group consisting of Atto590, Alexa594, STAR600, STAR 635P, STAR RED, Atto647N, Bodipy650-X, 580CP-methoxy, 610CP, SiR-methyl, sulfoCy5, IRDye800CW and indocyano green (ICG).

Thus, according to one preferred embodiment, Z is Atto590 (CAS no of NHS-ester 670269-33-7, wherein it is to be understood that upon linkage, the NHS—O group is absent in group Z), having the following structure, wherein the “*” indicates the connection site to L².

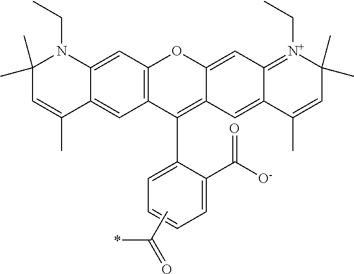

According to a further preferred embodiment, Z is Alexa594 (6-[2,4(or 2,5)-dicarboxyphenyl]-1,2,10,11-tetrahydro-1,2,2,10,10,11-hexamethyl-4,8-bis(sulfomethyl)-pyrano[3,2-g:5,6-g]diquinolin-13-ium), having the following structure, wherein the “*” indicates the connection site to L²;

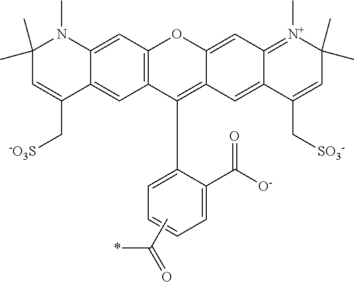

According to a further preferred embodiment, Z is STAR600 (available e.g. from Abberior), having the following structure, wherein the “R” represents a chemical bond to L².

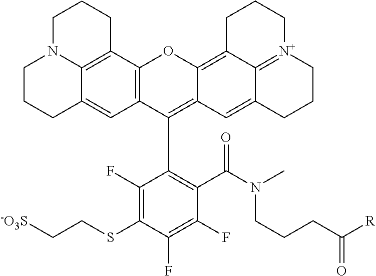

According to a further preferred embodiment, Z is STAR635P (available e.g. from Abberior, see Wurm, C. A., Kolmakov, K., Gottfert, F. et al. Novel red fluorophores with superior performance in STED microscopy. Opt Nano 1, 7 (2012)), having the following structure, wherein the “R” represents a chemical bond to L².

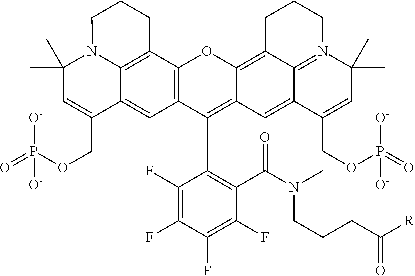

According to a further preferred embodiment, Z is STAR RED (available e.g. from Abberior, see Wurm, C. A., Kolmakov, K., Gottfert, F. et al. Novel red fluorophores with superior performance in STED microscopy. Opt Nano 1, 7 (2012)), having the following structure, wherein the “R” represents a chemical bond to L².

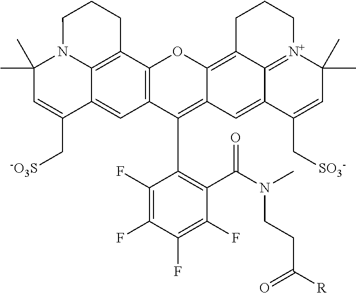

According to a further preferred embodiment, Z is ATTO647N, having the following structure, wherein the “*” represents a chemical bond to L²:

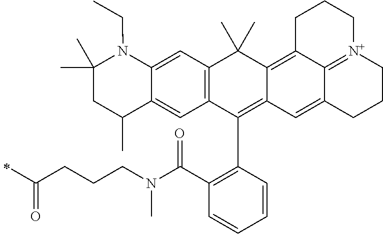

According to a further preferred embodiment, Z is Bodipy650-X (CAS 04-0; 1616842-78-4 of NHS-ester, wherein it is to be understood that upon linkage, the NHS—O group is absent in group Z, see Mitronova, G. Y., et al., Sci Rep 7, 12319 (2017)), having the following structure, wherein the “*” indicates the connection site to L²:

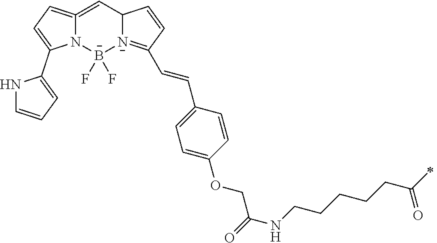

According to a further preferred embodiment, Z is 580CP-methoxy (N. Butkevich, G. Y. Mitronova, S. C. Sidenstein, J. L. Klocke, D. Kamin, D. N. H. Meineke, E. D'Este, P.-T. Kraemer, J. G. Danzl, V. N. Belov, S. W. Hell, Angew. Chem. Int. Ed. 2016, 55, 3290), having the following structure, wherein the “R” represents a chemical bond to L².

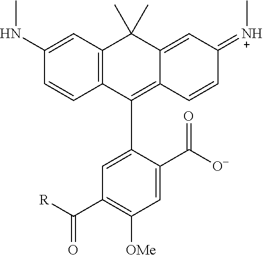

According to a further preferred embodiment, Z is 610CP (CAS 1877282-17-1, see N. Butkevich, G. Y. Mitronova, S. C. Sidenstein, J. L. Klocke, D. Kamin, D. N. H. Meineke, E. D'Este, P.-T. Kraemer, J G. Danzl, V. N. Belov, S. W. Hell, Angew. Chem. Int. Ed. 2016, 55, 3290), having the following structure, wherein the “R” represents a chemical bond to L²:

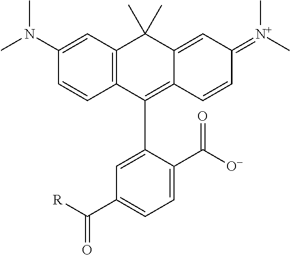

According to a further preferred embodiment, Z is SiR-methyl (see Lukinavioius, G., et al., Nature Chem 5, 132-139 (2013)) having the following structure, wherein the “R” represents a chemical bond to L².

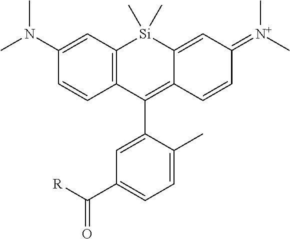

According to a further preferred embodiment, Z is IRDye800CW (CAS 1088919-86-1) having the following structure, wherein the “*” indicates the connection site to L²

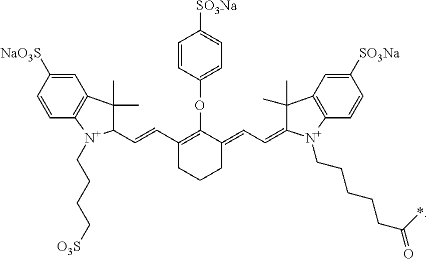

According to a further preferred embodiment, Z is ICG (3599-32-4) having the following structure.

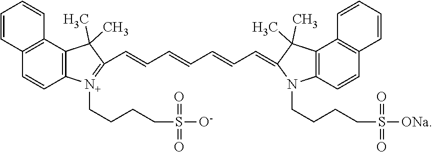

According to a further preferred embodiment, Z is sulfoCy5 (CAS 1144107-82-3 (potassium salt), CAS 1121756-16-8 (inner salt), CAS 2098639-31-5 (sodium salt)) having the structure —C(═O)—C¹ herein C¹ is

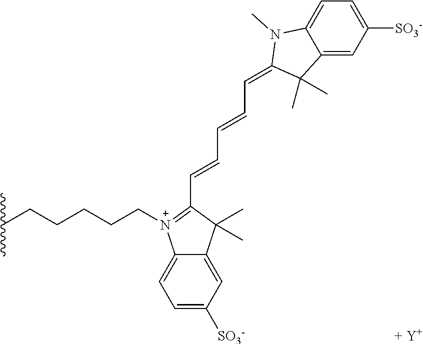

and wherein Y⁺ is a suitable pharmaceutical acceptable salt, preferably Na+ or K+, more preferably Na+.

According to a further preferred embodiment, the Z has the formula (C)

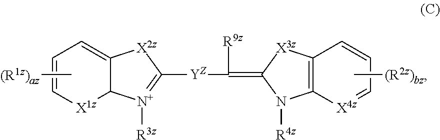

wherein
X^{1z} and X^{4z} are independently selected from the group consisting of —N=, —N(R^{5z})=, and —C(R^{6z})=;
X^{2z} and X^{3z} are independently selected from the group consisting of O, S, Se, N(R^{5z}), and C(R^{6z}R^{7z}), preferably both are C(CH₃)₂;
Y^{z} is a linker connecting the two moieties of (C) and permitting electron delocalization between said moieties, wherein Y^{z} optionally comprises a group (L^{z}-)_{c}Z⁰;
az and bz are independently selected from the group consisting of 1, 2, and 3;
each R^{1z} and each R^{2z} is independently selected from the group consisting of (L^{z}-)_{c}Z^{z}, (L^{z}-)_{c}Z⁰ and H; and two adjacent R^{1z} and/or two adjacent R^{2z} can also form an aromatic ring, which is optionally substituted with one or more (L^{z}-)_{c}Z^{z} or (L^{z}-)_{c}Z⁰;
R^{3z}, R^{4z}, R^{5z}, R^{6z}, R^{7z}, R^{9z} are independently selected from the group consisting of (L^{z}-)_{c}Z^{z}, (L^{z}-)_{c}Z⁰, and H;
each c is independently 0, or 1;
each L^{z} is independently T¹, —OT¹-, -ST¹-, —C(O)T¹-, —C(O)OT¹-, —OC(O)T¹-, —C(O)NHT¹-, —NHC(O)T¹, or a C₁₋₁₀ alkylene group, which is optionally interrupted and/or terminated by one or more of —O—, —S—, —C(O)—, —C(O)O—, —OC(O)—, —C(O)NH—, —NHC(O)O—, and T¹;
T¹ is phenyl, naphthyl, indenyl, indanyl, tetralinyl, decalinyl, adamantyl, C₃₋₇ cycloalkyl, 3 to 7 membered heterocyclyl, or 7 to 11 membered heterobicyclyl, wherein T¹ is optionally substituted with one or more substituents selected from the group consisting of halogen, CN, C(O)R^{8z}, COOR^{8z}, OR^{8z}, C(O)N(R^{8z}R^{8az}), S(O)₂N(R^{8z}R^{8az}), S(O)N(R^{8z}R^{8az}), S(O)₂R^{8z}, N(R^{8z})S(O)₂N(R^{8az}R^{8bz}), SR^{8z}, N(R^{8z}R^{8az}), NO₂; OC(O)R^{8z}, N(R_{8z})C(O)R^{8az}, N(R^{8z})S(O)₂R^{8az}, N(R^{8z})S(O)R^{8az}, N(R^{8z})C(O)N(R^{8az}R^{8bz}), N(R^{8z})C(O)OR^{8az}, OC(O)N(R^{8z}R^{8az}), oxo (═O), where the ring is at least partially saturated, or C₁₋₆ alkyl, wherein C₁₋₆ alkyl is optionally substituted with one or more halogen, which are the same or different;
each Z^{z} is independently H, halogen, CN, C(O)R^{8z}, C(O)OR^{8z}, C(O)O⁻ OR^{8z}, C(O)N(R^{8z}R^{8az}), S(O)₂OR^{8z}, S(O)₂O—, S(O)₂N(R^{8z}R^{8az}), S(O)N(R^{8z}R^{8az}), S(O)₂R^{8z}, S(O)R^{8z}, N(R^{z})S(O)₂N(R^{8az}R^{8bz}), SR^{8z}, N(R^{8z}R^{8az}), NO₂; P(O)(OR^{8z})₂, P(O)(OR^{8z})O⁻, OC(O)R^{8z}, N(R^{8z})C(O)R^{8az}, N(R^{8z})S(O)₂R^{8az}, N(R^{8z})S(O)R^{8az}, N(R^{8z})C(O)N(R^{8az}R^{8bz}), N(R^{8z})C(O)OR^{8az}, or OC(O)N(R^{8z}R^{8az});
R^{8z}, R^{8az}, R^{8bz} are independently selected from the group consisting of H, or C₁₋₆ alkyl, wherein C₁₋₆ alkyl is optionally substituted with one or more halogen, which are the same or different;
Z⁰ is a chemical bond connecting the dye Z to the group -(L^{BZ})n_{bz}-B, provided that one of R^{1z}, R^{2z}, R^{3z}, R^{4z}, R^{5z}, R^{6z}, R^{7z}, R^{9z} is (L^{z}-)_{c}Z⁰ or that Y^{z} comprises (L-)_{c}Z⁰.

The term “optionally substituted” means unsubstituted or substituted. Generally—but not limited to—, “one or more substituents” means one, two or three, preferably one or two substituents and more preferably one substituent. Generally these substituents can be the same or different. “Alkyl” means a straight-chain or branched hydrocarbon chain. Each hydrogen of an alkyl carbon may be replaced by a substituent as further specified herein. As used in this context of the application, the terms “alkyl”, “alkyl residue” and “alkyl group” and “alkyl moiety” may be understood as a straight-chain or branched saturated hydrocarbon chain. “Straight-chain” may be also designated as “unbranched” or “linear”. Preferably, the alkyl is a straight chain. As used in this context of the application, the term “alkylene” means a straight-chain or branched saturated hydrocarbon chain wherein two moieties of a molecule are linked by the alkylene residue. “Straight-chain” may be also designated as “unbranched” or “linear”. Each hydrogen of an alkylene carbon may or may not be replaced by a substituent (i.e., may be substituted or unsubstituted) as further specified herein).

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl, tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent as further specified herein. “C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl; tert-butyl, n-pentyl, n-hexyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₆ alkyl carbon may be replaced by a substituent as further specified herein. “C₁₋₈ alkylene residue” means an alkylene chain having 1-8 carbon atoms, e.g. —CH₂—, —CH₂—CH₂—, —CH(CH₃)—, —CH₂—CH₂—CH₂—, —CH(C₂H₅)—, —C(CH₃)₂—, —CH₂—C(CH₃)₂—, —C(CH₂—CH₃)₂—, —CH(CH₂—CH₃)—, —CH₂—CH(CH₃)(CH₂—CH₃)—, —CH(CH₃)(CH₂—CH₃)—, —(CH₂)₄—, —(CH₂)₅—, —(CH₂)₆—, —(CH₂)₇—, —(CH₂)₈—, etc., when two moieties of a molecule are linked by the alkylene group. The terms “C₄₋₈ alkylene” and “C₆ alkylene” are defined accordingly. The terms “C₃₋₇ alkylene” and “C₅ (C₄) alkylene” are defined accordingly. The term “C₁₋₁₀ alkylene group” means a bivalent straight-chain or branched hydrocarbon chain having 1 to 10 carbon atoms. Each hydrogen of an alkyl carbon may be replaced by a substituent as further specified herein. Examples are methylene (—CH₂—)—CH₂—CH₂—, —CH(CH₃)—, —CH₂—CH₂—CH₂—, —CH(C₂H₅)—, —C(CH₃)₂—. Each hydrogen of a C₁₋₁₀ alkylene group carbon may be replaced by a substituent as further specified herein. Accordingly, “C₁₋₁₀ alkylene residue” means an alkylene chain having 1-10 carbon atoms when two moieties of a molecule are linked by the alkylene group. Preferably, but not necessarily, the C₁₋₁₀ alkylene residue in the context of residue f of the spacer y is a straight-chain, i.e., unbranched, C₁₋₁₀ alkylene residue, in which optionally one or more hydrogen(s) are substituted and/or in which optionally one or more —CH₂— moieties may be replaced by —O— or —NH—. The expression “one or more —CH₂— moieties may optionally be replaced by” means that the indicated number of CH₂ groups can be replaced by an atom or group specified herein. In addition one or more hydrogens as specified herein can be replaced by a substituent. C₁₋₁₀ alkylene group “optionally interrupted and/or terminated” means that the alkylene chain is interrupted between two carbon atoms by an atom or a chemical group as specified herein or the alkylene group is terminated by said atom or group following the carbon at least at one end of the alkylene chain or the alkylene chain is both, interrupted and terminated or the alkylene chain is neither interrupted nor terminated. As an example without being bound to that example a C₃ alkylene group which is optionally interrupted and/or terminated with one or more X may have the sequence C—C—C, C—C—C—X, X—C—C—C, X—C—C—C—X, C—X—C—C, C—C—X—C, C—X—C—X—C, X—C—C—X—C, X—C—X—C—X—C, X—C—X—C—C—X, X—C—X—C—X—C—X.

The term “carbocycle” refers to a partly or fully saturated or aromatic carbocyclic mono-, bi- or tricyclic fused or unfused ring system. This includes phenyl and C₃₋₇ cycloalkyl rings. Preferred carbocycles having 5, 6 or 7 carbon atoms are cyclopentene, cyclohexene, phenyl, cycloheptane, especially cyclohexane. “C₃₋₇ cycloalkyl” or “C₃₋₇ cycloalkyl ring” means a cyclic alkyl chain having 3-7 carbon atoms, e.g. cyclopropyl, cyclobutyl, cyclopentyl, cyclohexyl, cyclohexenyl, cycloheptyl. Preferably, cycloalkyl refers to cyclopropyl, cyclobutyl, cyclopentyl, cyclohexyl, or cycloheptyl. Each hydrogen of a cycloalkyl carbon may be replaced by a substituent as further specified herein. The term “C₃₋₅ cycloalkyl” or “C₃₋₅ cycloalkyl ring” is defined accordingly. “Halogen” means fluoro, chloro, bromo or iodo. It is generally preferred that halogen is fluoro or chloro. Within the meaning of the present description the term “aromatic ring” means a carbocyclic or heterocyclic aromatic ring. Examples are benzene, naphthalene, 5 to 6 membered aromatic heterocycle and 9 to 11 membered aromatic heterobicyclyl. “3 to 7 membered heterocyclyl” or “3 to 7 membered heterocycle” as used in this context of the application means a ring with 3, 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 3 to 7 membered heterocycle are aziridine, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine. The term “4 to 7 membered heterocyclyl” or “4 to 7 membered heterocycle” is defined accordingly. The term “5 to 6 membered heterocyclyl” or “5 to 6 membered heterocycle” is defined accordingly. “5 to 6 membered aromatic heterocyclyl” or “5 to 6 membered aromatic heterocycle” means a heterocycle derived from cyclopentadienyl or benzene, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, triazole, tetrazole, pyridine, pyrimidine, pyridazine, pyrazine, triazine. “5 membered aromatic heterocyclyl” or “5 membered aromatic heterocycle” means a heterocycle derived from cyclopentadienyl, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, triazole, tetrazole. “7 to 11 membered heterobicyclyl” or “7 to 11 membered heterobicycle” means a heterocyclic system of two rings with 7 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 7 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine or pteridine. The term 7 to 11 membered heterobicycle also includes spiro structures of two rings like 6-oxa-2-azaspiro[3,4]octane, 2-oxa-6-azaspiro[3.3]heptan-6-yl or 2,6-diazaspiro[3.3]heptan-6-yl or bridged heterocycles like 8-aza-bicyclo[3.2.1]octane or 2,5-diazabicyclo[2.2.2]octan-2-yl or 3,8-diazabicyclo[3.2.1]octane. “9 to 11 membered aromatic heterobicyclyl” or “9 to 11 membered aromatic heterobicycle” means a heterocyclic system of two rings, wherein at least one ring is aromatic and wherein the heterocyclic ring system has 9 to 11 ring atoms, where two ring atoms are shared by both rings and that may contain up to the maximum number of double bonds (fully or partially aromatic) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 9 to 11 membered aromatic heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, dihydroquinoline, tetrahydroquinoline, isoquinoline, tetrahydroisoquinoline, dihydroisoquinoline, benzazepine, purine or pteridine. The terms “9 to 10 membered aromatic heterobicyclyl” or “9 to 10 membered aromatic heterobicycle” are defined accordingly.

Accordingly, one of the residues R^{1z}, R^{2z}, R^{3z}, R^{4z}, R^{5z}, R^{6z}, R^{7z} serve as connecting group, atom or bond (L^{z}-)_{c}Z⁰ of the dye Z to the group -(L^{BZ})n_{bz}-B

Preferably R³ or R⁴ represent (L^{z}-)_{c}Z⁰. More preferably, R^{3z} is (L^{z}-)_{c}Z⁰.

Preferably, in formula (C) X^{1z} and X^{4z} are the same and preferably C(R^{6z}), more preferably CH.

Preferably, in formula (C) X^{2z} and X^{3z} are the same and preferably C(R⁶R⁷), more preferably R⁶ and R⁷ are the same and even more preferably L^{z}-Z^{z} with L^{z}=C₁₋₁₀ alkylene, and even more preferably L^{z}=CH₂, and Z^{z}=H.

Preferably, R⁹ is H.

Preferably, in formula (C) Y^{z} does not comprise (L^{z}-)_{c}Z⁰ and preferably Y^{z} is

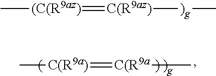

wherein g is 1, 2, 3, or 4 (preferably 2 or 3, more preferably 3) and each R^{9az} is (L^{z}-)_{c}Z^{z}, or H; and two R^{9az} can also form a carbocyclic ring having 5, 6, or 7 carbon atoms or a 4 to 7 membered heterocylic ring;
each c is independently 0, or 1;
each L^{z} is independently T¹, —OT¹-, -ST¹-, —C(O)T¹-, —C(O)OT¹-, —OC(O)T¹-, —C(O)NHT¹-, —NHC(O)T¹, or a C₁₋₁₀ alkylene group, which is optionally interrupted and/or terminated by one or more of —O—, —S—, —C(O)—, —C(O)O—, —OC(O)—, —C(O)NH—, —NHC(O)O—, and T¹;
T¹ is phenyl, naphthyl, indenyl, indanyl, tetralinyl, decalinyl, adamantyl, C₃₋₇ cycloalkyl, 4 to 7 membered heterocyclyl, or 7 to 11 membered heterobicyclyl, wherein Tⁱ is optionally substituted with one or more substituents selected from the group consisting of halogen, CN, C(O)R^{8z}, COOR^{8z}, OR^{8z}, C(O)N(R^{8z}R^{8az}), S(O)₂N(R^{8z}R^{8az}), S(O)N(R^{8z}R^{8az}), S(O)₂R^{8z}, N(R^{8z})S(O)₂N(R^{8az}R^{8bz}), SR^{8z}, N(R^{8z}R^{8az}), NO₂; OC(O)R^{8z}, N(R^{8z})C(O)R^{8az}, N(R^{8z})S(O)₂R^{8az}, N(R^{8z})S(O)R^{8az}, N(R^{8z})C(O)N(R^{8az}R^{8bz}), N(R^{8z})C(O)OR^{8az}, OC(O)N(R^{8z}R^{8az}), oxo (═O), where the ring is at least partially saturated, or C₁₋₆ alkyl, wherein C₁₋₆ alkyl is optionally substituted with one or more halogen, which are the same or different;
R^{8z}, R^{8az}, R^{8bz} are independently selected from the group consisting of H, or C₁₋₆ alkyl, wherein C₁₋₆ alkyl is optionally substituted with one or more halogen, which are the same or different;
each Z^{z} is independently H, halogen, CN, C(O)R^{8z}, C(O)OR^{8z}, C(O)O⁻ OR^{8z}, C(O)N(R^{8z}R^{8az}), S(O)₂OR^{8z}, S(O)₂O—, S(O)₂N(R^{8z}R^{8az}), S(O)N(R^{8z}R^{8az}), S(O)₂R^{8z}, S(O)R^{8z}, N(R^{8z})S(O)₂N(R^{8z}R^{8bz}), SR^{8z}, N(R^{8z}R^{8az}), NO₂; P(O)(OR^{8z})₂, P(O)(OR^{8z})O⁻, OC(O)R^{8z}, N(R^{8z})C(O)R^{8az}, N(R^{8z})S(O)₂R^{8az}, N(R^{8z})S(O)R^{8az}, N(R^{8z})C(O)N(R^{8az}R^{8bz}), N(R^{8z})C(O)OR^{8az}, or OC(O)N(R^{8z}R^{8az}).

Preferably, Y^{z} is

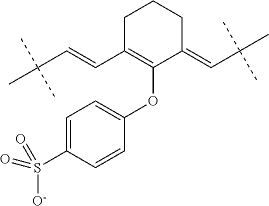

or —(C(R^{9az})═C(R^{9az})—)_{g}—
with g=2 and each R^{9az}=H.

Preferably, in formula (C) az and bz are the same and preferably 1, more preferably with R^{1z} and R^{2z}=SO₃⁻.

Preferably, in formula (C) az and bz are the same and 2, preferably wherein two adjacent R^{1z} and two adjacent R^{2z} form a phenyl ring.

Preferably, in formula (C) one of R^{3z} and R^{4z} is (L^{z}-)_{c}Z^{Z0} and the other is (L^{z}-)_{c}Z^{z} with L^{z}- ═C₁₋₁₀ alkylene and Z^{z}=H or SO₃⁻ and c preferably =1.

Preferably, (L^{z}-)_{c}Z⁰ is C₁₋₁₀ alkylene-C(O)— (c=1, Z⁰ a chemical bond), preferably C₃₋₇ alkylene-C(O)—, more preferably C₅ alkylene-—C(O)—, connecting (C) to to the group -(L^{BZ})n_{bz}-B.

In a highly preferred embodiment, the has the structure —C(═O)—C¹ and C¹ is selected from the group consisting of the following structures.

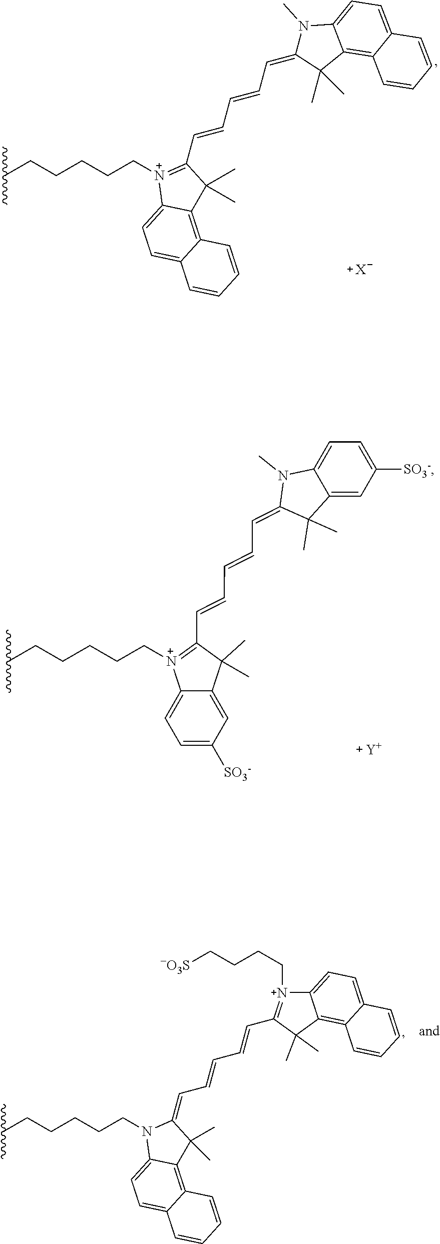

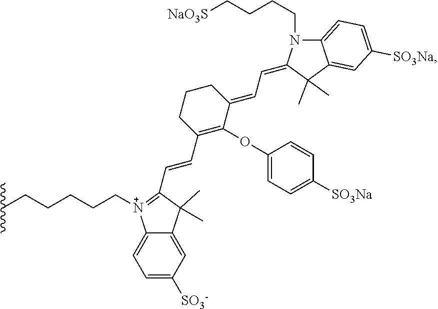

wherein X⁻ is a pharmaceutically acceptable negatively charged counterion;
wherein Y⁺ is a pharmaceutically acceptable positively charged counterion; and
wherein the wavy line indicates the conjugation site to the group —C(═O), which group is attached to the group -(L^{BZ})n_{bz}-B.

Most preferred Z is —C(═O)—C¹ with C¹ being

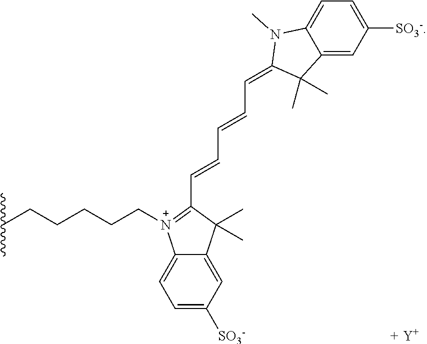

And with Y+ being a pharmaceutically acceptable counterion, as described above and below.

Preferably the dye is sulfoCy5.

A pharmaceutically acceptable negatively charged counterion X⁻ may be understood in the broadest sense as laid out above. Likewise, also a pharmaceutically acceptable positively charged counterion Y⁺ may have any valency. Therefore, Y⁺ may exemplarily have a charge of +1, +2, +3 or +4, preferably of +1 or +2. Y⁺ may be any pharmaceutically acceptable positively charged ion. Preferably, the ion is such well-soluble in aqueous liquids. Exemplarily, Y⁺ may be selected from the group consisting of a cation of an alkali metal (e.g., Na⁺, K⁺, Li⁺), a cation of an alkaline earth metal (e.g., Mg²⁺, Ca²⁺), Al³⁺, NH₄⁺, H⁺ and a cation of an organically bound amine. Further, it will be understood that the counterion typically depends on the surrounding liquids such as those comprised in the buffer the compound is dissolved in and the body fluids after injection in vivo. In vivo, extracellularly, one of the main, but not sole positively charged counterions is Na⁺.

Preferably, the PSMA ligand has a structure is selected from the structures shown in FIG. 1, FIG. 2, and FIG. 3.

Most preferably, the PSMA ligand has the structure as shown in FIG. 2

### The Complex

As described above, the present invention also relates to a complex comprising
(a) a radionuclide, and
(b) a PSMA binding ligand, as described above or below, or a pharmaceutically acceptable salt or solvate thereof.

Typical pharmaceutically acceptable salts include those salts prepared by reaction of the PSMA binding ligands of the present invention with a pharmaceutically acceptable mineral or organic acid or an organic or inorganic base. Such salts are known as acid addition and base addition salts. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulfonic acid. Salts of amine groups may also comprise quaternary ammonium salts in which the amino nitrogen carries a suitable organic group such as an alkyl, alkenyl, alkynyl, or aralkyl moiety. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred. It should be recognized that the particular counter ion forming a part of any salt of this invention is usually not of a critical nature, so long as the salt as a whole is pharmacologically acceptable and as long as the counter ion does not contribute undesired qualities to the salt as a whole.

The term “pharmaceutically acceptable solvate” encompasses also suitable solvates of the PSMA binding ligands of the invention, wherein the PSMA binding ligand combines with a solvent such as water, methanol, ethanol, DMSO, acetonitrile or a mixture thereof to form a suitable solvate such as the corresponding hydrate, methanolate, ethanolate, DMSO solvate or acetonitrilate.

### The Radionuclide

Depending on whether the PSMA binding ligands of the invention are to be used as radio-imaging agents or radio-pharmaceuticals different radionuclides are complexed to the chelator. The complexes of invention may contain one or more radionuclides, preferably one radionuclide. These radionuclides are preferably suitable for use as radio-imaging agents or as therapeutics for the treatment of proliferating cells, for example, PSMA expressing cancer cells, in particular PSMA-expressing prostate cancer cells. According to the present invention they are called “metal complexes” or “radiopharmaceuticals”.

Preferred imaging methods are positron emission tomography (PET) or single photon emission computed tomography (SPECT).

Preferably, the at least one radionuclide is selected from the group consisting ⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰u, ²²³P, ¹⁶⁵Er, ⁵²Fe, ⁵⁹Fe and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb).

More preferably, the at least one radionuclide is selected from the group consisting ⁹⁰Y, ⁶⁸Ga, ¹⁷⁷Lu, ²²⁵Ac, and ²¹³Bi. More preferably, the radionuclide is ¹⁷⁷Lu or ²²⁵Ac.

Preferably, the radionuclide has a half-life of at least 30 min, more preferably of at least 1 h, more preferably at least 12 h, even more preferably at least Id, most preferably at least 5 d; also preferably, the radionuclide has a half-life of at most 1 year, more preferably at most 6 months, still more preferably at most 1 month, even more preferably at most 14 d. Thus, preferably, the radionuclide has a half-life of from 30 min to 1 year, more preferably of 12 h to 6 months, even more preferably of from 1 d to 1 month, most preferably of from 5 d to 14 d.

Preferably, the radionuclide is an α- and/or β-emitter, i.e. the radionuclide preferably emits α-particles (α-emitter) and/or β-radiation (β-emitter).

Preferably, in case the radionuclide is an α-emitter, the α-particle has an energy of from 1 to 10 MeV, more preferably of from 2 to 8 MeV, most preferably of from 4 to 7 MeV.

Preferably, in case the radionuclide is a β-emitter, the β-radiation has an energy of from 0.1 to 10 MeV, more preferably of from 0.25 to 5 MeV, most preferably of from 0.4 to 2 MeV.

Preferred radionuclides emitting β-radiation are selected from the group consisting of ⁹⁰Y, ¹⁷⁷Lu, ⁵⁹Fe, ⁶⁶Cu, ⁶⁷Cu, ¹⁶¹Tb, ¹⁵³Sm, ²¹²Pb, ²¹¹Pb, ²¹³Pb, ²¹⁴Pb, ²⁰⁹Pb Very preferred radionuclides emitting β-radiation are ¹⁷⁷Lu or ⁹⁰Y, most preferably ¹⁷⁷Lu. Preferably in this case the use is diagnosis or therapy.

Preferred radionuclides emitting α-radiation are e.g. selected from the group consisting of ²¹³Bi, ²²⁵Ac, ¹⁴⁹Tb, ²³⁰U and ²²³Ra. ²¹³Bi, ²³⁰U, more preferably the radionuclide is ²²⁵Ac and/or ²¹³Bi. A very preferred radionuclide emitting α-radiation is e.g. ²²⁵Ac. Preferably in this case the use is therapy.

According to a further embodiment, the radionuclide is a positron emitter. In this case the radionuclide is preferably selected from the group consisting ⁸⁹Zr, ⁴⁴Sc, ⁶⁶Ga ⁶⁸Ga and ⁶⁴Cu. In this case, the use is preferably PET diagnosis.

According to a further preferred embodiment, radionuclide is a gamma emitter. In this case the radionuclide is preferably selected from the group consisting ¹¹¹In, ⁶⁷Ga, ^{99m}Tc, ¹⁵⁵Tb, ¹⁶⁵Er and ²⁰³Pb. In this case, the use preferably is SPECT diagnosis.

According to a further preferred embodiment, the radionuclide emits Auger electrons, and preferably decays by electron capture. In this case, the radionuclide is preferably selected from the group consisting of ⁶⁷Ga, ¹⁵⁵Tb, ¹⁵³Gd, ¹⁶⁵Er and ²⁰³Pb. In this case, the use is preferably therapy.

### Pharmaceutical Composition

As described above, the present invention also relates to a pharmaceutical composition comprising the PSMA binding ligand as described above or below, or a complex as described above or below. It is to be understood that the pharmaceutical compositions preferably comprise therapeutically effective amounts of the PSMA binding ligand and/or the complex, respectively. The pharmaceutical composition may further comprise at least one organic or inorganic solid or liquid and/or at least one pharmaceutically acceptable carrier.

The terms “medicament” and “pharmaceutical composition”, as used herein, relate to the PSMA binding ligands and/or complexes of the present invention and optionally one or more pharmaceutically acceptable carrier, i.e. excipient. The PSMA binding ligands of the present invention can be formulated as pharmaceutically acceptable salts; salts have been described herein above. The pharmaceutical compositions are, preferably, administered locally (e.g. intra-tumorally), topically or systemically. Suitable routes of administration conventionally used for drug administration are oral, intravenous, or parenteral administration as well as inhalation. A preferred route of administration is parenteral administration. A “parenteral administration route” means modes of administration other than enteral and topical administration, usually by injection, and includes, without limitation, intravenous, intramusclular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Preferably, administration is by intravenous administration or infusion. However, depending on the nature and mode of action of a PSMA binding ligand, the pharmaceutical compositions may be administered by other routes as well.

Moreover, the PSMA binding ligands can be administered in combination with other drugs either in a common pharmaceutical composition or as separated pharmaceutical compositions wherein said separated pharmaceutical compositions may be provided in form of a kit of parts. The PSMA binding ligands are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The excipient(s) must be acceptable in the sense of being compatible with the other ingredients of the formulation and, within the scope of sound medical judgment, suitable for use in contact with the tissues of a patient without excessive toxicity, irritation, allergic response, or other problem or complication, commensurate with a reasonable benefit/risk ratio. Preferably, an excipient is being not deleterious to the recipient thereof. The excipient employed may be, for example, a solid, a gel or a liquid carrier. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, non-immunogenic stabilizers and the like. When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The readymade solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

As will be understood by the skilled person, the dosage of the PSMA binding ligand depends on a variety of factors. As is well known in the medical arts, dosages for any one patient may depend upon many factors, including the patient's size, body surface area, age, the particular PSMA binding ligand to be administered, sex, time and route of administration, general health, and other drugs being administered concurrently. Moreover, the dosage of the PSMA binding ligand will typically also depend on the intended application, e.g. diagnostic and/or therapeutic use, and on the method of detection used, if any.

In diagnostic uses, in particular administration of a labeling dose is preferred, the term “labeling dose” relating to a dose of the PSMA binding ligand enabling labeling PSMA-expressing tissue, preferably, specifically labeling PSMA-expressing, more preferably enabling differentiation between cancerous tissue and non-cancerous tissue. In case the PSMA binding ligand is detected via its dye moiety, e.g. during surgery, preferably, the labeling dose is at least 200 μg, preferably at least 500 μg, more preferably at least 1 mg of labeling compound per subject. Preferably, the labeling dose is of from 0.2 mg to 100 mg, more preferably is of from 0.5 to 25 mg, still more preferably is of from 1 mg to 10 mg, most preferably is of from 1 mg to 5 mg, of said labeling compound per subject. Thus, the labeling dose may e.g. be about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, or about 1.5 mg, per subject. The aforesaid labeling doses preferably are labeling doses for human subjects. Also preferably, the labeling dose preferably is of from 3 μg/kg body mass to 300 μg/kg body mass, more preferably of from 5 μg/kg body mass to 100 μg/kg body mass, even more preferably of from 7.5 μg/kg body mass to 50 μg/kg body mass, still more preferably about 15 μg/kg body mass. Also preferably, the dose is a single dose, i.e. exactly one dose, preferably administered within a time frame of at least twelve hours, preferably at least one day, more preferably at least two days; thus, in the aforesaid time frame, preferably no further dose of labeling compound is administered. Preferably, said labeling dose is administered to the patient at of from 0.25 h to 48 h before diagnosis and/or detecting the PSMA binding ligand. In case the PSMA binding ligand is a complex as described elsewhere herein and is detected in a diagnostic application via a bound radionuclide, the dose is calculated as an activity dose as specified elsewhere herein. In terms of compound dose, the dose will strongly depend on the specific activity of the radionuclide, and may generally be approx. of from 5fold to 1000fold lower compared to the diagnostic doses specified herein above for detection via the dye moiety.

A therapeutically effective dose refers to an amount of the PSMA binding ligand to be used in a pharmaceutical composition of the present invention which prevents, ameliorates, or treats the symptoms accompanying a disease or condition referred to in this specification. Therapeutic efficacy and toxicity of such PSMA binding ligands can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. As is understood by the skilled person, doses in radiotherapy typically are indicated as activity doses, as specified elsewhere herein. Progress can be monitored by periodic assessment.

The pharmaceutical compositions and formulations referred to herein are administered at least once in order to treat or prevent a disease or condition recited in this specification. However, the said pharmaceutical compositions may be administered more than one time, for example from one to ten times. Preferably, the pharmaceutical compositions may be administered at a frequency of once every one to six months, more preferably once every two to four months. Specific pharmaceutical compositions are prepared in a manner well known in the pharmaceutical art and comprise at least one active PSMA binding ligand referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

The term “patient”, as used herein, relates to a vertebrate, preferably a mammalian animal, more preferably a human, monkey, cow, horse, cat or dog. Preferably, the mammal is a primate, more preferably a monkey, most preferably a human).

The dosage of the PSMA binding ligand administered to a patient, preferably, is defined as a compound dosage, i.e. the amount of PSMA binding ligand administered to the patient. Preferred diagnostic compound dosages are total doses of 1-10 nmol/patient; thus, preferably, the diagnostic compound dosage is of from 0.02 to 0.1 nmol/kg body weight. Preferred therapeutic compound dosages are total doses of 10 to 100 nmol/patient; thus, preferably, the therapeutic compound dosage is of from 0.2 to 1 nmol/kg body weight.

As will be understood by the skilled person, the dosage of the complex as specified herein, i.e. a complex comprising, preferably consisting of, a radionuclide and a PSMA binding ligand, preferably is indicated as compound dosage as specified above, preferred dosages being the same as specified above. More preferably, the dosage of the complex is indicated as activity dosage, i.e. as the amount of radioactivity administered to the patient. Preferably, the activity dosage is adjusted such as to avoid adverse effects as specified elsewhere herein. Preferably, a patient-specific dose, preferably a patient-specific activity dosage, is determined taking into account relevant factors as specified elsewhere herein, in particular taking into account therapeutic progress and/or adverse effects observed for the respective patient. Thus, preferably, the activity dosage is adjusted such that the organ-specific dose in salivary glands is at most 30 Sv, more preferably less than 20 Sv, still more preferably less than 10 Sv, most preferably less than 5 Sv.

The effective amount may be administered once (single dosage) with an activity dosage of from about 2 MBq to about 30 MBq, preferably 4 to 30 Mbq, more preferably 6 to 30 Mbq, more preferably 8 to 30 Mbq, more preferably 10 to 30 Mbq, more preferably 15 to 30 Mbq, preferably 20 to 30 Mbq to the patient. Thus, a preferred therapeutic dose in such case is of from 2 MBq to about 30 MBq/patient, preferably 4 to 30 Mbq/patient, more preferably 6 to 30 Mbq/patient, more preferably 8 to 30 Mbq/patient, more preferably 10 to 30 Mbq/patient, more preferably 15 to 30 Mbq/patient, preferably 20 to 30 Mbq/patient. Preferably said activity dosage ranges from about 10 to 30 MBq per administration, such as for example about 10, 11, 12, 13, 14, 15, 16, 17, 18, 19, 20, 21, 22, 23, 24, 25, 26, 27, 28, 29 or 30 MBq, or any range between any two of the above values. However, as specified herein below, depending on the type of radiation emitted by the radionuclide and/or on the application, higher or lower doses may be envisaged. The phrases “effective amount” or “therapeutically-effective amount” as used herein mean that amount of a PSMA binding ligand, material, or composition comprising a PSMA binding ligand of the invention, or other active ingredient which is effective for producing some desired therapeutic effect in at least a sub-population of cells in a patient at a reasonable benefit/risk ratio applicable to any medical treatment. A therapeutically effective amount with respect to a PSMA binding ligand of the invention means that amount of therapeutic agent alone, or in combination with other therapies, that provides a therapeutic benefit in the treatment or prevention of a disease. Used in connection with a PSMA binding ligand of the invention, the term can encompass an amount that improves overall therapy, reduces or avoids symptoms or causes of disease, or enhances the therapeutic efficacy of or synergies with another therapeutic agent.

According to a preferred embodiment, the radionuclide is a β-emitter as specified herein above, more preferably is ¹⁷⁷Lu and the use is diagnosis; in such case, the activity dosage of the complex preferably is at least 100 kBq/kg body weight, more preferably at least 500 kBq/kg body weight, most preferably at least 1 MBq/kg body weight. More preferably the radionuclide is a β-emitter as specified herein above, more preferably is ¹⁷⁷Lu and the use is therapy, preferably therapy of prostate carcinoma as specified elsewhere herein; in such case, the activity dosage of the complex preferably is at least 25 MBq/kg body weight, more preferably at least 50 MBq/kg body weight, most preferably at least 80 MBq/kg body weight. Thus, a preferred therapeutic dose in such case is of from 2 to 10 Gbq/patient, more preferably of from 4 to 8 GBq/patient, most preferably is about 6 GBq/patient.

More preferably, the radionuclide is an α-emitter as specified herein above, more preferably is ²²⁵Ac and the use is therapy, preferably therapy of prostate carcinoma as specified elsewhere herein; in such case, the activity dosage of the complex is preferably in the range of from 25 kBq/kg to about 500 kBq/kg of body weight of said patient, more preferably, the activity dosage of the complex is at least 75 kBq/kg body weight, more preferably at least 100 kBq/kg body weight, still more preferably at least 150 kBq/kg body weight, most preferably at least 200 kBq/kg body weight. Thus, preferably, in such case, the activity dosage of the complex is of from 75 to 500 kBq/kg body weight, more preferably of from 100 to 400 kBq/kg body weight, still more preferably of from 150 to 350 kBq/kg body weight, most preferably of from 200 to 300 kBq/kg body weight. The present invention also relates to a PSMA binding ligand as described above or below, a complex as described above or below, or a pharmaceutical composition as described herein above, for use in diagnosis, preferably for diagnosing a cell proliferative disease or disorder, in particular prostate cancer and/or metastases thereof. Further, the present invention also relates to a PSMA binding ligand as described above or below a complex as described above or below, or a pharmaceutical composition as described above or below, for use in medicine, preferably for treating or preventing a cell proliferative disease or disorder, in particular prostate cancer and/or metastases thereof.

The term “diagnosing”, as used herein, refers to assessing whether a subject suffers from a disease or disorder, preferably cell proliferative disease or disorder, or not. As will be understood by those skilled in the art, such an assessment, although preferred to be, may usually not be correct for 100% of the investigated subjects. The term, however, requires that a, preferably statistically significant, portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05. As will be understood by the skilled person, diagnosing may comprise further diagnostic assessments, such as visual and/or manual inspection, determination of tumor biomarker concentrations in a sample of the subject, X-ray examination, and the like. The term includes individual diagnosis of as well as continuous monitoring of a patient. Monitoring, i.e. diagnosing the presence or absence of cell proliferative disease or the symptoms accompanying it at various time points, includes monitoring of patients known to suffer from cell proliferative disease as well as monitoring of subjects known to be at risk of developing cell proliferative disease. Furthermore, monitoring can also be used to determine whether a patient is treated successfully or whether at least symptoms of cell proliferative disease can be ameliorated over time by a certain therapy. Moreover, the term also includes classifying a subject according to a usual classification scheme, e.g. the T1 to T4 staging, which is known to the skilled person. In accordance, diagnosing may include determining number and location of metastases, affected lymph nodes, and the like.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, as specified herein above. The term “preventing” and “prevention” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the PSMA binding ligand according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed herein above.

Preferably, treatment and/or prevention comprises administration of at least one PSMA binding ligand and/or at least one complex as specified elsewhere herein, more preferably at an activity dosage and/or compound dosage as specified above.

The term “cell proliferative disease”, as used herein, relates to a disease of an animal, including man, characterized by uncontrolled growth by a group of body cells (“cancer cells”). This uncontrolled growth may be accompanied by intrusion into and destruction of surrounding tissue (infiltration) and possibly spread of cancer cells to other locations in the body (metastasis).

Preferably, also included by the term cancer is a relapse. Thus, preferably, the cancer is a solid cancer, a metastasis, or a relapse thereof. Preferably, the cell proliferative disease is an uncontrolled proliferation of cells comprising cells expressing PSMA.

Thus, preferably, the cell proliferative disease is a PSMA expressing cancer. The term “PSMA expressing cancer” refers to any cancer whose cancerous cells express Prostate Specific Membrane Antigen (PSMA). Preferably cancers (or cancer cells) that may be treated according to the invention are selected among prostate cancer, conventional renal cell cancers, cancers of the transitional cells of the bladder, lung cancers, testicular-embryonal cancers, neuroendocrine cancers, colon cancers, brain tumors and breast cancers, more preferably are selected among PSMA-positive prostate cancer, PSMA-positive renal cell cancers, PSMA-positive cancers of the transitional cells of the bladder, PSMA-positive lung cancers, PSMA-positive testicular-embryonal cancers, PSMA-positive neuroendocrine cancers, PSMA-positive colon cancers, PSMA-positive brain tumors, and PSMA-positive breast cancers. Whether a cancer is PSMA-positive can be established by the skilled person by methods known in the art, e.g. in vitro by immunostaining of a cancer sample, or in vivo e.g. by PSMA scintigraphy, preferably both as described in Kratochwil et al. (2017, J Nucl Med 58(10):1624. In particularly preferred aspects of the invention, said PSMA expressing cancer is prostate cancer or breast cancer, more preferably prostate cancer; and even more preferably advanced-stage prostate cancer. Thus, preferably, the cell proliferative disease is prostate cancer stage T2, more preferably stage T3, most preferably stage T4. Preferably, the cell proliferative disease is metastatic prostate cancer, more preferably is metastatic castration-resistant prostate cancer. Advantageously, it has been shown in the studies underlying the present invention that administration of the PSMA binding ligand and/or complexes of the present invention to a patient results in an improved pharmacokinetic profile, in particular improved renal excretion with essentially unchanged enrichment in target tissue, preferably cell proliferative tissue, more preferably cancer tissue, as compared to e.g. the meanwhile commonly used PSMA-617. Due to the improved excretion, adverse side effects on non-target tissues, in particular the salivary and/or lacrimal glands, can be avoided and/or reduced. This is advantageous, because the adverse side effects on the salivary glands are considered as dosage-limiting (cf. Kratochwil et al. (2017, J Nucl Med 58(10):1624). Based on the finding of the present invention, larger amounts of compounds and/or complexes and in particular higher doses of radioactivity can be administered to a patient as compared to the compounds and complexes described in the art. Thus, the therapeutic window is broader than with the compounds presently in use. Also advantageously, the PSMA binding ligands of the present invention provide for improved diagnosis, since the co-labelling of irrelevant tissue and organs, in particular salivary glands, lacrimal glands and/or kidneys, is reduced. Also, by means of the improved renal excretion, background and/or false-positive labeling in diagnstic applications is reduced, preferably leading to a clearer identification of PSMA-expressing tissues.

Thus, the PSMA binding ligands and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein the PSMA binding ligands and/or complexes display an advantageous renal excretion profile, preferably with a favorable clearance acceleration. Thus, adverse side effects on the patient's kidney are diminished. Thus, the present invention also relates to a PSMA binding ligands and/or complexes of the present invention or a pharmaceutical composition, as described above, for treating and/or preventing PSMA expressing cancer, in particular prostate cancer and/or metastases thereof, in a patient in need thereof, the subject suffering from renal failure.

As detailed herein above and in the Examples, the compounds as specified herein provide for accelerated excretion while maintaining essentially the same enrichment in target tissue as e.g. PSMA-617, so adverse effects on non-target tissues, preferably the salivary and/or lacrimal glands, are avoided or reduced. Thus, treatment and/or diagnosis as specified herein has less or less severe adverse side effects, e.g. on the salivary glands and/or lacrimal glands, or is preferably not accompanied by adverse side effects, in particular on the salivary glands and/or lacrimal glands.

Preferably, the PSMA binding ligands of the present invention allow for reduction and/or avoidance of adverse side effects, e.g. on the salivary glands and/or lacrimal glands, while maintaining therapeutic efficacy essentially unchanged. As will be understood by the skilled person in view of the above, the PSMA binding ligands as specified herein preferably further make a use of higher concentrations of the compounds and/or higher doses of radioactivity feasible while at least not increasing adverse effects, which may be particularly useful in diagnostic applications to detect e.g. small metastasis or small amounts of remaining tumor tissue, and/or in treatment.

Accordingly, the PSMA binding ligands and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein xerostomia is avoided.

Preferably the PSMA binding ligand, as described above or below, or the complex, as described above or below, or the pharmaceutical composition, as described above or below, are used for in vivo imaging and radiotherapy. Suitable pharmaceutical compositions may contain a radio imaging agent, or a radiotherapeutic agent that has a radionuclide either as an element, i.e. radioactive iodine, or a radioactive metal chelate complex of the PSMA binding ligand in an amount sufficient for imaging, together with a pharmaceutically acceptable radiological vehicle. The radiological vehicle should be suitable for injection or aspiration, such as human serum albumin; aqueous buffer solutions, e.g., tris(hydromethyl)-aminomethane (and its salts), phosphate, citrate, bicarbonate, etc; sterile water physiological saline; and balanced ionic solutions containing chloride and or dicarbonate salts or normal blood plasma cautions such as calcium potassium, sodium and magnesium.

The concentration of the imaging agent or the therapeutic agent in the radiological vehicle should be sufficient to provide satisfactory imaging. Appropriate dosages have been described herein above. The imaging agent or therapeutic agent should be administered so as to remain in the patient for about 1 hour to 10 days, although both longer and shorter time periods are acceptable. Therefore, convenient ampoules containing 1 to 10 mL of aqueous solution may be prepared.

Imaging may be carried out in a manner known to the skilled person, for example by injecting a sufficient amount of the imaging composition to provide adequate imaging and then scanning with a suitable imaging or scanning machine, such as a tomograph or gamma camera. In certain embodiments, a method of imaging a region in a patient includes the steps of: (i) administering to a patient a diagnostically effective amount of a PSMVA binding ligand complexed with a radionuclide; (ii) exposing a region of the patient to the scanning device; and (ii) obtaining an image of the region of the patient. In certain embodiments of the region imaged is the head or thorax. In other embodiments, the PSMA binding ligands and complexes target the PSMA protein. Thus, in some embodiments, a method of imaging tissue such as spleen tissue, kidney tissue, or PSMA-expressing tumor tissue is provided including contacting the tissue with a complex synthesized by contacting a radionuclide and PSMA binding ligand, as described above.

The amount of the PSMA binding ligand of the present invention, or a formulation comprising a complex of the PSMA binding ligand, or its salt, solvate, stereoisomer, or tautomer that is administered to a patient depends on several physiological factors. These factors are known by the physician, including the nature of imaging to be carried out, tissue to be targeted for imaging or therapy and the body weight and medical history of the patient to be imaged or treated using a radiopharmaceutical.

Further, the present invention relates to PSMA binding ligand and/or a complex as described above or below as a labeling agent for detecting cancerous tissue in a subject.

The term “labeling agent” is understood by the skilled person. Preferably, the term relates to a compound labeling PSMA-expressing tissue, more preferably, specifically labeling PSMA-expressing cancer tissue. The term “specific labeling” of a PSMA-expressing tissue preferably relates to a labeling which enables differentiation between PSMA-expressing tissue and non-PSMA-expressing tissue, such as e.g. cancer-adjacent tissue. Preferably, said differentiation is enabled in vitro and/or in vivo, more preferably in vivo. Thus, preferably, the labeling enables differentiation between adjacent cancerous and non-cancerous tissues, e.g. in an operation area. Thus, specific labeling does not necessarily have to enable differentiation between PSMA-expressing tissue and any non-PSMA-expressing tissue in a subject; it, preferably, is sufficient if PSMA-expressing tissue can be differentiated from non-PSMA-expressing tissue in the vicinity of PSMA-expressing tissue. Thus, the differentiation may preferably be between cancerous tissue and non-cancerous tissue in the abdominal cavity, more preferably within at most 10 cm, more preferably at most 5 cm, even more preferably at most 2 cm from a cancerous tissue. Also, it preferably is not required that differentiation is enabled from easily identifiable intact structures in the subject, e.g. the kidney, the liver, and the like. The differentiation is preferably made by visual inspection, e.g. by a medical practitioner, or may be assisted or made by an optical device adapted to detect the label comprised in the labeling compound. Preferably, specific labeling is labeling of PSMA-expressing tissue, preferably of cancer cells, more intensely by a factor of at least 2, more preferably at least 5, even more preferably at least 10, still more preferably at least 25, more preferably at least 100, compared to non-PSMA-expressing tissue. The labeling agent may be the PSMA binding ligand described herein or a metal chelate thereof, in particular a radiometal chelate thereof.

Also, the present invention relates to a PSMA binding ligand, a complex, or a pharmaceutical composition, all as specified herein above, for use in fluorescence guided surgery.

In view of the above, the invention also provides a method for treating a patient by administering to a patient a therapeutically effective amount of a complex, as described above or below, to treat a patient suffering from a cell proliferative disease or disorder. Specifically, the cell proliferative disease or disorder to be treated or imaged using a PSMA binding ligand, pharmaceutical composition or radiopharmaceutical in accordance with this invention is a cancer, for example, prostate cancer and/or prostate cancer metastasis in e.g. lung, liver, kidney, bones, brain, spinal cord, bladder, etc.

The PSMA binding ligands of the invention may e.g. be synthesized in solution as well as on solid phase using e.g. standard peptide coupling procedures, such as Fmoc solid phase coupling procedures. Preferably, the chelator is coupled to the remaining part of the molecule in the last coupling step followed by a deprotection step and in case of solid phase chemistry, cleavage from the resin. However, other synthetic procedures are possible and known to the skilled person. A preferred synthesis of the PSMA binding ligands of the present invention is described in detail in the example section.

Also, the present invention relates to a method for labeling PSMA-expressing tissue in a subject, comprising administering a PSMA binding ligand to said subject.

The present invention also relates to a method for identifying PSMA-expressing tissue tissue in a subject comprising (a) labeling PSMA-expressing tissue by administering a PSMA binding ligand to said subject; and (b) identifying labeled PSMA-expressing tissue in situ; and to a method for removing cancerous tissue from a subject, said method comprising (a) labeling cancerous tissue by administering a PSMA binding ligand to said subject; (b) identifying labeled PSMA-expressing tissue in situ; and (c) removing said cancerous tissue.

Further, the present invention relates to a use of a PSMA binding ligand as specified herein above in the manufacture of a diagnostic composition for labeling PSMA-expressing tissue, preferably in vivo labeling cancerous tissue, and/or for the manufacture of a therapeutic composition for treatment of a PSA-expressing cellular proliferation.

Summarizing the findings of the present invention, the following embodiments are preferred:
1. PSMVA binding ligand or a pharmaceutically acceptable salt or solvate thereof comprising a PSMA binding motif Q, a chelator residue A, a dye group Z and at least one linker L^{BQ} comprising at least one amino acid X₁, wherein
   X₁ is preferably a neutral amino acid, or
   X₁ is preferably a N-alkylated amino acid, preferably N-methylated amino acid, more preferably X₁ is a neutral N-alkylated amino acid, more preferably a neutral N-methylated amino acid, more preferably wherein X₁ is —N(CH₃)—CH₂—C(═O)—.
2. PSMA binding ligand according to embodiment 1 having the structure

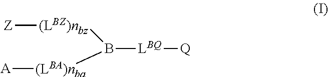

wherein Q is the PSMA binding motif
   A is the chelator residue,
   Z is the dye group,
   B is a branching group,
   L^{BQ} is a linker connecting Q with B, said linker comprising the at least one amino acid X₁,
   L^{BZ} is a linker, wherein n_{bz} is 0 or 1
   And L^{BA} is a linker, wherein n_{ba} is 0 or 1.
3. PSMA binding ligand according to embodiment 2, wherein B has the structure

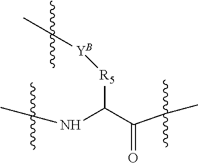

wherein R₅ is an alkyl group, preferably a —(CH₂)₂₋₄ group, and Y^{B} is a functional group linking R₅ to the group -(L^{BZ})n_{bz}-Z or -(L^{BA})n_{ba}-A, wherein Y^{B} is preferably a group —NH— or —C(═O)—.
4. PSMA binding ligand according to embodiment 4, having one of the following structures (Ia) or (Ib), preferably the structure (Ia)

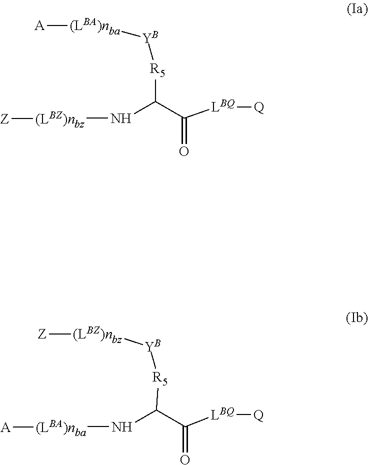

5. PSMA binding ligand according to any one embodiment 1 to 5 or a pharmaceutically acceptable salt or solvate thereof, the PSMA binding motif Q having the structure

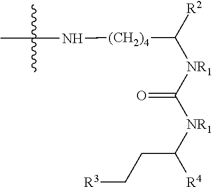

wherein R¹ is H or —CH₃, preferably H, wherein R², R³ and R⁴ are independently of each other, selected from the group consisting of—CO₂H, —SO₂H, —SO₃H, —OSO₃H, —PO₂H, —PO₃H and —OPO₃H₂.
6. PSMA binding ligand according to any one of embodiment 1 or 5 or a pharmaceutically acceptable salt or solvate thereof,
   wherein A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid](NOPO), 3,6,9,15-tetraazabicyclo[9.3.1]pentadeca-1 (15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA).
7. The PSMA binding ligand according to any one of embodiments 1 to 6 or a pharmaceutically acceptable salt or solvate thereof, wherein A is a chelator residue having a structure selected from the group consisting of

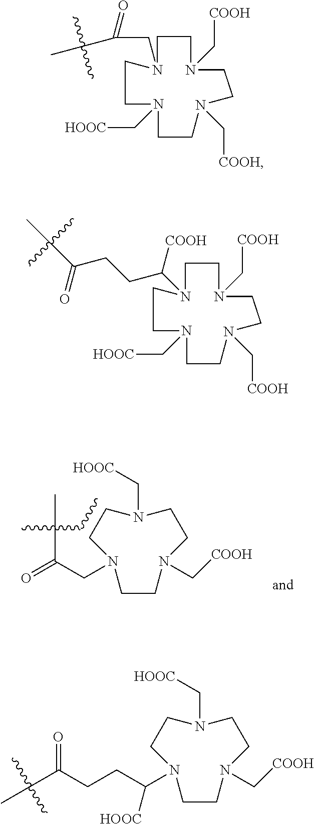

8. The PSMA binding ligand according to any one of embodiments 1 to 7 or a pharmaceutically acceptable salt or solvate thereof, wherein A is a chelator residue having the structure

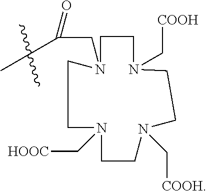 [SMILES: *C(=O)CN1CCCN(CC(=O)O)CCN(CC(=O)O)CCN(CC(=O)O)CC1]

9. The PSMA binding ligand according to embodiment 7 or 8, having the structure (Ia)

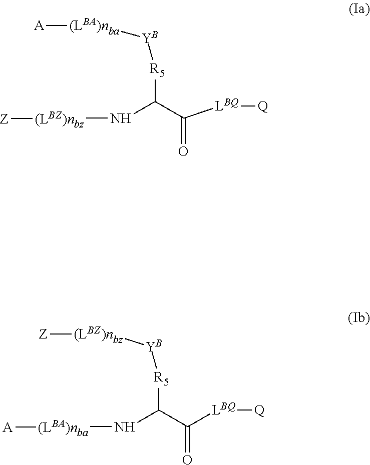

wherein R₅ is an alkyl group, preferably a —(CH₂)₁₋₁₀ group, more preferably a —(CH₂)₂₋₄ group, and Y^{B} is a functional group linking R₅ to the group -(L^{BZ})n_{bz}-Z or -(L^{BA})n_{ba}-A, wherein Y^{B} is preferably a group —NH— or —C(═O)—, more preferably —NH—.
10. The PSMA binding ligand according to any one of embodiment 1 to 9, wherein the dye group Z is a fluorescent dye comprising a fluorophore with excitation and emission spectra in the range of about 350 to 100 nm, preferably 400 nm to about 850 nm.
11. The PSMA binding ligand according to any one of embodiments 1 to 10 or a pharmaceutically acceptable salt or solvate thereof, wherein the dye group Z is a fluorescent dye Z is selected from the group consisting of AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750, Cy2, Cy3, Cy3B, Cy3.5, Cy5, sulfoCy5, Cy5.5 and Cy7, DyLight 350, DyLight 405, DyLight 488, DyLight 550, DyLight 594, DyLight 633, DyLight 650, DyLight 680, DyLight 750 and DyLight 800, FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid), 7-Hydroxy-4-methylcoumarin-3, 7-Hydroxycoumarin-3-carboxylic acid (Pubchem SID 135727263), MCA (7-Methoxycoumarin-4-acetic acid) (Pubchem CID 342221), 7-Methoxycoumarin-3, AMF (4′-(Aminomethyl)fluorescein), 5-DTAF (5-(4,6-Dichlorotriazinyl)aminofluorescein), 6-DTAF (6-(4,6-Dichlorotriazinyl)aminofluorescein), 6-FAM (6-Carboxyfluorescein), 5 (6)-FAM cadaverine (Fluorescein-5 (6)-carboxamide cadaverine), 5-FAM cadaverine (Fluorescein-5-carboxamide cadaverine), 5 (6)-FAM ethylenediamine (Fluorescein-5 (6)-carboxamide ethylenediamine), 5-FAM ethylenediamme (Fluorescein-5 (6)-carboxamide ethylenediamine), 5-FITC (FITC Isomer I; fluorescein-5-isothiocyanate), 5-FITC cadaverin; Fluorescein-5-maleimide; 5-IAF (5-Iodoacetamidofluorescein), 6-JOE (6-Carboxy-4′,5′-dichloro-2′,7′-dimethoxyfluorescein), 5-CR110 (5-Carboxyrhodamine 110), 6-CR110 (6-Carboxyrhodamine 110), 5-CR6G (5-Carboxyrhodamine 6G); 6-CR6G (6-Carboxyrhodamine 6G), 5 (6)-Caroxyrhodamine 6G cadaverine; 5 (6)-Caroxyrhodamine 6G ethylenediamme, 5-ROX (5-Carboxy-X-rhodamine), 6-ROX (6-Carboxy-X-rhodamine); 5-TAMRA (5-Carboxytetramethylrhodamine), 6-TAMRA (6-Carboxytetramethylrhodamine), 5-TAMRA cadaverine, 6-TAMRA cadaverine, 5-TAMRA ethylenediamine (5-Carboxytetramethylrhodamine ethylenediamine), 6-TAMRA ethylenediamine (6-Carboxytetramethylrhodamine ethylenediamine), 5-TMR C6 maleimide, 6-TMR C6 maleimide, TR C2 maleimide, TR cadaverine, 5-TRITC (Tetramethylrhodamine-5-(and-6)-isothiocyanate), 6-TRITC, R isomer (Tetramethylrhodamine-6-isothiocyanate), Dansyl cadaverine (5-Dimethylaminonaphthalene-1-(N-(5-aminopentyl))sulfonamide), EDANS C2 maleimide (5-((2-Aminoethyl)amino)naphthalene-1-sulfonic acid C2 maleimide), EDANS acid (5-((2-Aminoethyl)amino)naphthalene-1-sulfonic acid); fluorescamine (4-Phenylspiro-[furan-2 (3H),1-phthalan]-3,3′-dion), NBD (4-Chlor-7-nitro-benzo-2-oxa-1,3-diazol), pyrromethene, Texas Red (1H,5H,11H,15H-Xantheno[2,3,4-ij5,6,7-i′j′]diquinolizin-18-ium, 9-[2(or 4)-(chlorosulfonyl)-4(or 2)-sulfophenyl]-2,3,6,7,12,13,16,17-octahydro-, inner salt), Cy5, Cy5 succinimidylester (3H-Indolium, 2-[5-[1-[6-[(2,5-dioxo-1-pyrrolidinyl)oxy]-6-oxohexyl]-1,3-dihydro-3,3-dimethyl-5-sulfo-2H-indol-2-ylidene]-1,3-pentadien-1-yl]-1-ethyl-3,3-dimethyl-5-sulfo-, inner salt), Cy5 sacridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, 2-aminonaphthalen, indicyano green (ICG), IRDye800CW, Bodipy650-X, CF680R, 580CP-methocy, 610CP, SiR-methyl, ATTO 390, ATTO 425, ATTO 465, ATTO 488, ATTO 495, ATTO 514, ATTO 520, ATTO 532, ATTO Rho6G, ATTO 550, ATTO 565, ATTO Rho3B, ATTO Rho11, ATTO Rho12, ATTO Thio12, ATTO Rho101, ATTO 590, ATTO Rho13, ATTO 594, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO647N, STAR 600, STAR635 P, STAR RED, 580CP-methoxy and derivatives thereof, preferably Z is selected from the group consisting of Atto590, Alexa594, STAR600, STAR 635P, STARRED, Atto647N, Bodipy650-X, 580CP-methoxy, 610CP, SiR-methyl, sulfoCy5, IRDye800CW and indocyano green (ICG) and derivatives thereof, more preferably Z is sulfoCy5.
12. The PSMVA binding ligand according to any one of embodiments 1 to 10,
   having the structure

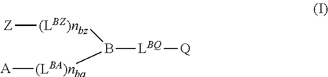

wherein Q is the PSMA binding motif
   A is the chelator residue,
   Z is the dye group,
   B is a branching group,
   L^{BQ} is a linker connecting Q with B, said linker comprising the at least one amino acid X₁,
   L^{BZ} is a linker, wherein n_{bz} is 0 or 1
   and L^{BA} is a linker, wherein n_{ba} is 0 or 1,
   and wherein the dye group Z is a fluorescent dye Z comprising, preferably consisting of, the structure

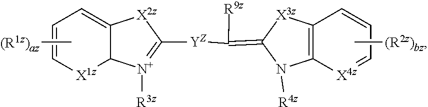

wherein
   X^{1z} and X^{4z} are independently selected from the group consisting of —N=, —N(R^{5z})=, and —C(R^{6z})=;
   X^{2z} and X^{3z} are independently selected from the group consisting of O, S, Se, N(R^{Sz}), and C(R^{6z}R^{7z}), preferably both are C(CH₃)₂;
   Y^{z} is a linker connecting the two moieties of (C) and permitting electron delocalization between said moieties, wherein Y^{z} optionally comprises a group (L^{z}-)_{c}Z⁰;
   az and bz are independently selected from the group consisting of 1, 2, and 3;
   each R^{1z} and each R^{2z} is independently selected from the group consisting of (L^{z}-)_{c}Z^{z}, (L^{z}-)_{c}Z⁰ and H; and two adjacent R^{1z} and/or two adjacent R^{2z} can also form an aromatic ring, which is optionally substituted with one or more (L^{z}-)Z^{z} or (L^{z}-)_{c}Z⁰;
   R^{3z}, R^{4z}, R^{5z}, R^{6z}, R^{7z}, R^{9z} are independently selected from the group consisting of (L^{z}-)_{c}Z^{z}, (L^{z}-)_{c}Z⁰, and H;
   each c is independently 0, or 1;
   each L^{z} is independently T¹, —OT¹-, -ST¹-, —C(O)T¹-, —C(O)OT¹-, —OC(O)T¹-, —C(O)NHT¹-, —NHC(O)T¹, or a C₁₋₁₀ alkylene group, which is optionally interrupted and/or terminated by one or more of —O—, —S—, —C(O)—, —C(O)O—, —OC(O)—, —C(O)NH—, —NHC(O)O—, and T¹;
   T¹ is phenyl, naphthyl, indenyl, indanyl, tetralinyl, decalinyl, adamantyl, C₃₋₇ cycloalkyl, 3 to 7 membered heterocyclyl, or 7 to 11 membered heterobicyclyl, wherein T¹ is optionally substituted with one or more substituents selected from the group consisting of halogen, CN, C(O)R^{8z}, COOR^{8z}, OR^{8z}, C(O)N(R^{8z}R^{8az}), S(O)₂N(R^{8z}R^{8az}), S(O)N(R^{8z}R^{8az}), S(O)₂R^{8z}, N(R^{8z})S(O)₂N(R^{8z}R^{8bz}), SR^{8z}, N(R^{8z}R^{8az}), NO₂; OC(O)R^{8z}, N(R^{8z})C(O)R^{8az}, N(R^{8z})S(O)₂R^{8az}, N(R^{8z})S(O)R^{8az}, N(R^{8z})C(O)N(R^{8az}R^{8bz}), N(R^{8z})C(O)OR^{8az}, OC(O)N(R^{8z}R^{8az}), oxo (═O), where the ring is at least partially saturated, or C₁₋₆ alkyl, wherein C₁₋₆ alkyl is optionally substituted with one or more halogen, which are the same or different;
   each Z^{z} is independently H, halogen, CN, C(O)R^{8z}, C(O)OR^{8z}, C(O)O⁻ OR^{8z}, C(O)N(R^{8z}R^{8az}), S(O)₂OR⁸z, S(O)₂O⁻, S(O)₂N(R^{8z}R^{8az}), S(O)N(R^{8z}R^{8az}), S(O)₂R^{8z}, S(O)R^{8z}, N(R^{8z})S(O)₂N(R^{8az}R^{8bz}), SR^{8z}, N(R^{8z}R^{8az}), NO₂; P(O)(OR^{8z})₂, P(O)(OR^{8z})O⁻, OC(O)R^{8z}, N(R^{8z})C(O)R^{8az}, N(R^{8z})S(O)₂R^{8az}, N(R^{8z})S(O)R^{8az}, N(R^{8z})C(O)N(R^{8az}R^{8bz}), N(R^{8z})C(O)OR^{8az}, or OC(O)N(R^{8z}R^{8az});
   R^{8z}, R^{8az}, R^{8bz} are independently selected from the group consisting of H, or C₁₋₆ alkyl, wherein C₁₋₆ alkyl is optionally substituted with one or more halogen, which are the same or different;
   Z⁰ is a chemical bond connecting the dye Z to the group -(L^{BZ})n_{bz}-B,
   provided that one of R^{1z}, R^{2z}, R^{3z}, R^{4z}, R^{5z}, R^{6z}, R^{7z}, R^{9z} is (L^{z}-)_{c}Z⁰ or that Y^{z} comprises (L-)_{c}Z⁰.
13. The PSMA binding ligand according to any one of embodiment 12, wherein the dye group Z has the structure —C(═O)—C¹ and C¹ is selected from the group consisting of the following structures.

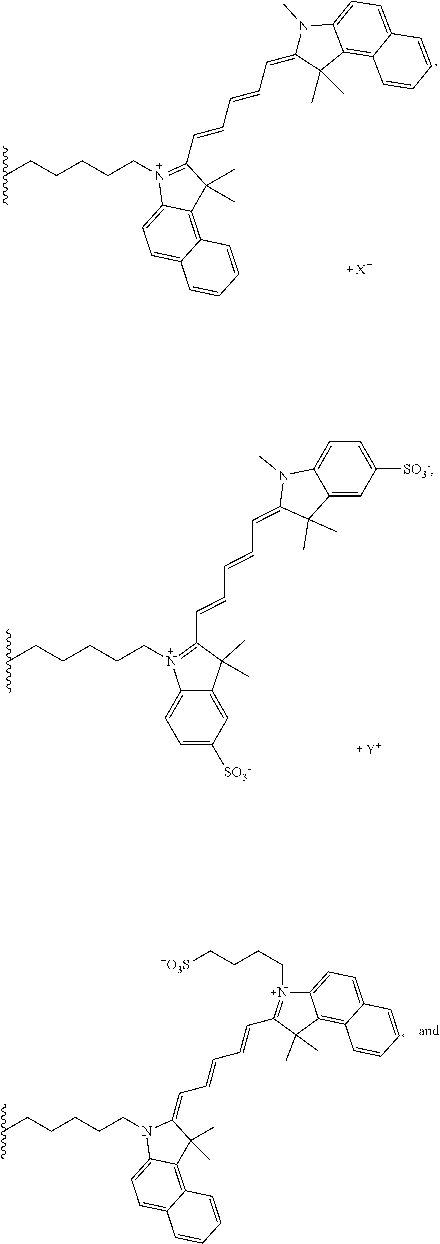

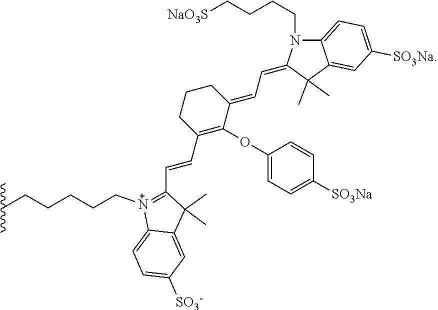

wherein X⁻ is a pharmaceutically acceptable negatively charged counterion; and wherein
   Y* is a pharmaceutically acceptable positively charged counterion
14. PSMA binding ligand according to any one of embodiments 1 to 8 or a pharmaceutically acceptable salt or solvate thereof, the PSMA ligand, preferably the linker L^{BQ}, further comprising at least one amino acid building block AS^{a}, wherein AS^{a} has the structure

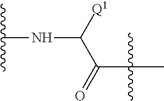

wherein Q¹ is selected from the group consisting of alkylaryl, arylalkyl, aryl, alkylheteroaryl, heteroarylalkyl and heteroaryl.
15. PSMA binding ligand according to any one of embodiments 1 to 9 or a pharmaceutically acceptable salt or solvate thereof, the PSMA ligand, preferably the linker L^{BQ}, comprising at least one amino acid building block AS^{b}, wherein AS^{b} has the structure (b)

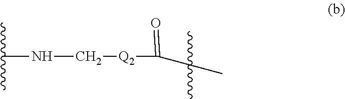

wherein Q² is selected from the group consisting of aryl, alkylaryl, arylalkyl, cycloalkyl, heterocycloalkyl, heteroaryl, heteroarylalkyl and alkylheteroaryl, preferably wherein Q² is

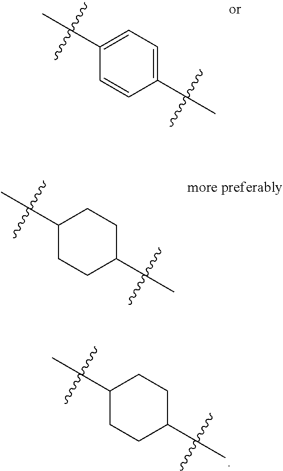 [SMILES: *c1ccc(*)cc1 ; *C1CCC(*)CC1 ; *C1CCC(*)CC1]

16. PSMVA binding ligand according to any one of embodiments 1 to 10 or a pharmaceutically acceptable salt or solvate thereof, the linker L^{BQ} further comprising at least one amino acid building block AS^{a} and at least one amino acid building block AS^{b}, wherein AS^{a} has the structure

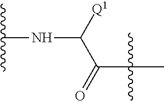

wherein Q¹ is selected from the group consisting of alkylaryl, arylalkyl, aryl, alkylheteroaryl, heteroarylalkyl and heteroaryl, and wherein AS^{b} has the structure (b)

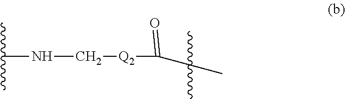

wherein Q² is selected from the group consisting of aryl, alkylaryl, arylalkyl, cycloalkyl, heterocycloalkyl, heteroaryl, heteroarylalkyl and alkylheteroaryl, preferably wherein Q² is

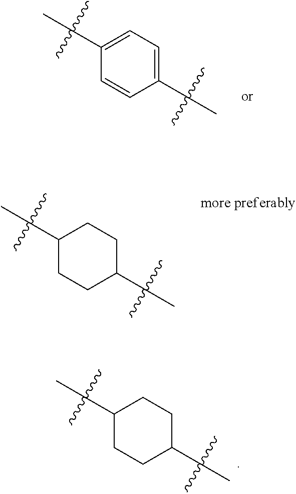 [SMILES: *c1ccc(*)cc1 ; *C1CCC(*)CC1 ; *C1CCC(*)CC1]

preferably wherein the linker L^{BQ} further comprises the linking unit -(AS^{b})_{q}-AS^{a}—, wherein q is an integer of from 0-3, preferably wherein q is 1.
17. PSMVA binding ligand according to any one of embodiments 1 to 11 or a pharmaceutically acceptable salt or solvate thereof, wherein Q is linked to -(AS^{b})_{q}-AS^{a}-, the PSMA binding ligand comprising the building block

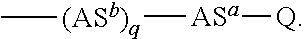

18. PSMA binding ligand according to embodiment 12 or a pharmaceutically acceptable salt or solvate thereof, the building block -L^{BQ}-Q comprising, preferably consisting of, the building block —(X¹)ₙ₁-(AS^{b})_{q}-AS^{a}-Q having the structure
19.

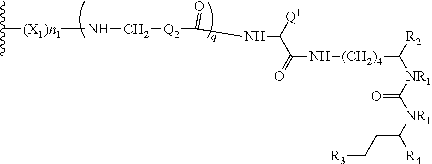

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂,
   Q¹ is selected from the group consisting of alkylaryl, arylalkyl, aryl, alkylheteroaryl, heteroarylalkyl and heteroaryl,
   Q² is selected from the group consisting of aryl, alkylaryl, arylalkyl, cycloalkyl, heterocycloalkyl, heteroaryl, heteroarylalkyl and alkylheteroaryl,
   and wherein q is an integer of from 0-3,
   and wherein X¹ is a methylated amino acid, preferably wherein X¹ is —N(CH₃)—CH₂—C(═O)— and n1 is an integer of from 0 to 25, preferably 5 to 15, more preferably 5 or 10, in particular 10.
20. The PSMA binding ligand according to embodiment 13 or a pharmaceutically acceptable salt or solvate thereof, wherein Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

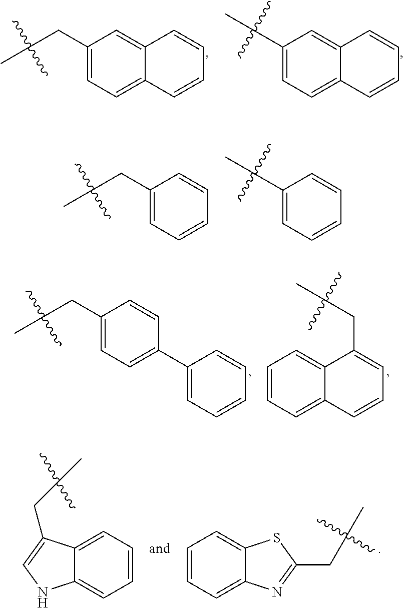

preferably wherein Q¹ is

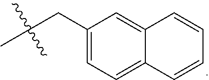 [SMILES: *Cc1ccc2ccccc2c1]

21. The PSMA binding ligand of any of one of embodiments 13 or 14, wherein R³, R² and R⁴ are —CO₂H and R¹ is H.
22. The PSMA binding ligand of any one of embodiments 13 to 15, wherein Q² is

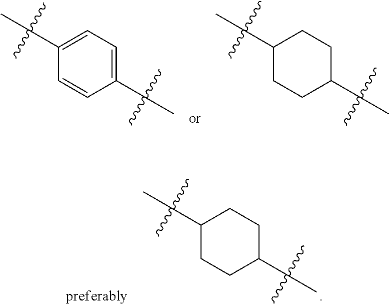 [SMILES: *c1ccc(*)cc1 ; *C1CCC(*)CC1 ; *C1CCC(*)CC1]

23. The PSMA binding ligand of any one of embodiment 1 to 22, the ligand having the structure

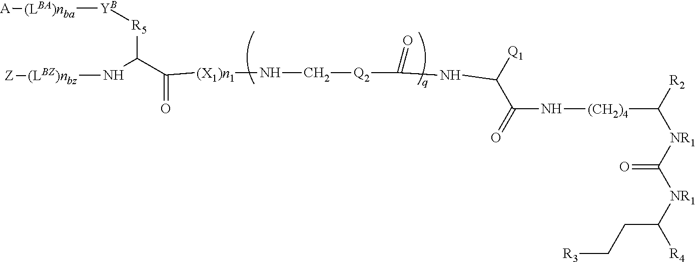

wherein R¹ is H or —CH₃, preferably H, wherein R², R³ and R⁴ are independently of each other, selected from the group consisting of—CO₂H, —SO₂H, —SO₃H, —OSO₃H, —PO₂H, —PO₃H and —OPO₃H₂,
   Q¹ is selected from the group consisting of alkylaryl, arylalkyl, aryl, alkylheteroaryl, heteroarylalkyl and heteroaryl,
   Q² is selected from the group consisting of aryl, alkylaryl, arylalkyl, cycloalkyl, heterocycloalkyl, heteroaryl, heteroarylalkyl and alkylheteroaryl,
   and wherein q is an integer of from 0-3,
   wherein X¹ is a methylated amino acid, preferably wherein X¹ is —N(CH₃)—CH₂—C(═O)— and n1 is an integer of from 1 to 25, wherein
   Z is the dye group,
   L^{BZ} is a linker, wherein n_{bz} is 0 or 1
   and L^{BA} is a linker, wherein n_{ba} is 0 or 1, preferably 0,
   and wherein R₅ is an alkyl group, preferably a —(CH₂)₂₋₄ group, and Y^{B} is a functional group linking R₅ to the group -(L^{BA})n_{ba}-A, wherein Y^{B} is preferably a group —NH— or —C(═O)—, more preferably a —NH— group.
24. The PSMA binding ligand of any one of embodiment 1 to 22, the ligand having the structure

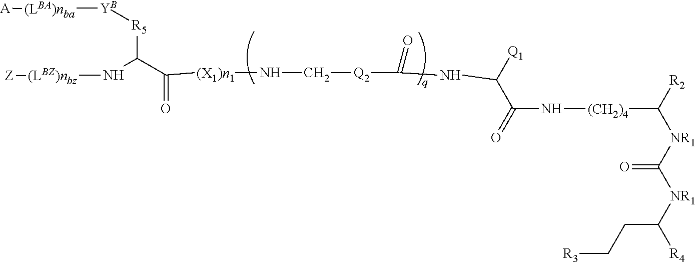

wherein R¹ is H or —CH₃, preferably H, wherein R², R³ and R⁴ are independently of each other, selected from the group consisting of—CO₂H, —SO₂H, —SO₃H, —OSO₃H, —PO₂H, —PO₃H and —OPO₃H₂,
Q¹ is selected from the group consisting of alkylaryl, arylalkyl, aryl, alkylheteroaryl, heteroarylalkyl and heteroaryl,
Q² is selected from the group consisting of aryl, alkylaryl, arylalkyl, cycloalkyl, heterocycloalkyl, heteroaryl, heteroarylalkyl and alkylheteroaryl,
and wherein q is an integer of from 0-3.
wherein X¹ is a methylated amino acid, preferably wherein X¹ is —N(CH₃)—CH₂—C(═O)— and n1 is an integer of from 1 to 25
and wherein
Z is the dye group,
L^{B}Z is a linker, wherein n_{bz} is 0 or 1
and L^{BA} is a linker, wherein n_{ba} is 0 or 1,
and wherein R₅ is an alkyl group, preferably a —(CH₂)₂₋₄ group, and Y^{B} is a functional group linking R₅ to the group -(L^{BZ})n_{bz}-Z, wherein Y^{B} is preferably a group —NH— or —C(═O)—, more preferably a —NH— group.
25. The PSMA binding ligand of any one of embodiment 23 or 24, wherein the L^{BQ} and/or L^{Bz} comprises at least one methylated amino acid.
26. The PSMA binding ligand of embodiment 23, the ligand having the structure (IIIa)

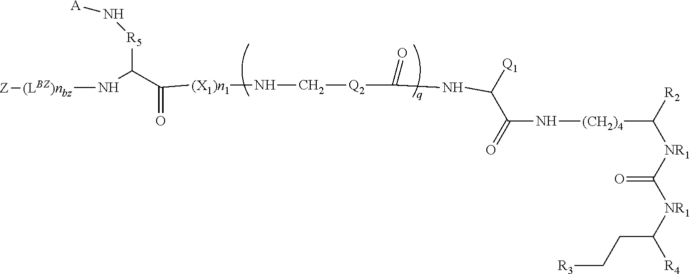

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂,
   Q¹ is selected from the group consisting of alkylaryl, arylalkyl, aryl, alkylheteroaryl, heteroarylalkyl and heteroaryl,
   Q² is selected from the group consisting of aryl, alkylaryl, arylalkyl, cycloalkyl, heterocycloalkyl, heteroaryl, heteroarylalkyl and alkylheteroaryl,
   and wherein q is an integer of from 0-3,
   wherein X¹ is a methylated amino acid, preferably wherein X¹ is —N(CH₃)—CH₂—C(═O)— and n1 is an integer of from 1 to 25, preferably 5 to 10, more preferably 10,
   and wherein
   Z is the dye group,
   L^{BZ} is a linker, wherein n_{bz} is 0 or 1
   and wherein R_{S} is an alkyl group, preferably a —(CH₂)₂₋₄ group.
27. The PSMA binding ligand of embodiment 26, wherein L^{BZ} comprises an amino acid sequence, and wherein Z is attached to the N-terminal end of L^{BZ} or to a functional group of a side chain of an amino acid present in L^{BZ}, with the proviso that in case L^{BZ} is attached to a side chain, the N-terminus is preferably acylated with a group —C(═O)—Rz, with R^{z} being preferably an C10-C20-alkyl chain.
28. The PSMA binding ligand of embodiment 26, wherein the amino acid sequence has structure —(X^{2bz})_{n2bz}—(X^{1bz})_{n1bz}— and Z is attached to the N-terminal end, and wherein X^{1z} is a methylated amino acid, preferably wherein X^{1z} is —N(CH₃)—CH₂—C(═O)— and n1bz is of from 1 to 20, preferably 4 to 10, more preferably 5, and wherein X^{2bz} is a neutral amino acid, preferably beta-Ala and n2bz is of from 1 to 20, preferably 1.
29. The PSMA binding ligand of embodiment 26, wherein the amino acid sequence has structure —(X^{3bz})_{n3bz}—(X^{1bz})_{n1bz}— and wherein X^{1bz} is a methylated amino acid, preferably wherein X^{1bz} is —N(CH₃)—CH₂—C(═O)— and n1z is of from 1 to 20, preferably 4 to 10, more preferably 5, and wherein X^{2bz} is glutamic acid or lysine, preferably lysine and Z is attached to the side chain of X^{2bz}, and n2bz is of from 1 to 20, preferably 1, and wherein the N terminus is preferably acylated.
30. The PSMA binding ligand of any one of embodiment 24, the ligand having the structure (IIIb)

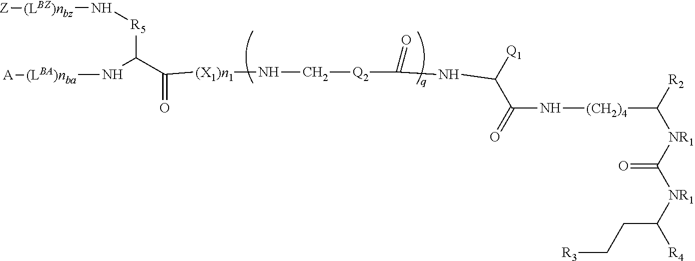

wherein R¹ is H or —CH₃, preferably H, wherein R², R³ and R⁴ are independently of each other, selected from the group consisting of—CO₂H, —SO₂H, —SO₃H, —OSO₃H, —PO₂H, —PO₃H and —OPO₃H₂,
   Q¹ is selected from the group consisting of alkylaryl, arylalkyl, aryl, alkylheteroaryl, heteroarylalkyl and heteroaryl,
   Q² is selected from the group consisting of aryl, alkylaryl, arylalkyl, cycloalkyl, heterocycloalkyl, heteroaryl, heteroarylalkyl and alkylheteroaryl,
   and wherein q is an integer of from 0-3.
   wherein X¹ is a methylated amino acid, preferably wherein X¹ is —N(CH₃)—CH₂—C(═O)— and n1 is an integer of from 1 to 25
   and wherein
   Z is the dye group,
   L^{BZ} is a linker, wherein n_{bz} is 0 or 1, preferably 0,
   and L^{BA} is a linker, wherein n_{ba} is 0 or 1,
   and wherein Rₛ is an alkyl group, preferably a —(CH₂)₂₋₄ group.
31. The PSMA binding ligand of embodiment 30, wherein L^{BA} comprises an amino acid sequence, and wherein A is attached to the N-terminal end of L^{BA} or to a functional group of a side chain of an amino acid present in L^{BA}, with the proviso that in case L^{BA} is attached to a side chain, the N-terminus is preferably acylated with a group —C(═O)—R^{z}, with R^{z} being preferably an C10-C20-alkyl chain.
32. The PSMA binding ligand of embodiment 31, and wherein the amino acid sequence has structure —(X^{2ba})_{n2ba}—(X^{1ba})_{n1ba}— and A is attached to the N-terminal end, and wherein X^{1ba} is a methylated amino acid, preferably wherein X^{1ba} is —N(CH₃)—CH₂—C(═O)— and n1ba is of from 1 to 20, preferably 4 to 10, more preferably 5, and wherein X^{2ba} is a neutral amino acid, preferably beta-Ala and n2ba is of from 1 to 20, preferably 1.
33. The PSMA ligand of embodiment 1 having the structure as shown in FIG. 1, FIG. 2 or FIG. 3, preferably as shown in FIG. 2
34. Complex comprising
   (a) a radionuclide, and
   (b) the PSMA binding ligand of any one of embodiments 1 to 33 or a pharmaceutically acceptable salt or solvate thereof.
35. The complex of embodiment 34, wherein, the radionuclide is selected from the group consisting ⁸⁹Zr, ⁴⁴Sc, ¹¹¹In, ⁹⁰Y, ⁶⁶Ga, ⁶⁷G, ⁶⁸G, ¹⁷⁷u, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb).
36. A pharmaceutical composition comprising a PSMA binding ligand of any one of embodiment 1 to 33 or a complex of embodiment 34 or 35.
37. A PSMA binding ligand of any one of embodiments 1 to 33, a complex of embodiment 34 or 35, or a pharmaceutical composition of claim 36 for use in medicine, preferably for treating and/or preventing PSMA expressing cancer, in particular prostate cancer and/or metastases thereof.
38. The PSMA binding ligand, complex, or pharmaceutical composition for use of embodiment 37, wherein adverse side effects on the kidney are reduced and/or avoided.
39. The complex or the pharmaceutical composition for use of embodiment 37 or 38, wherein the radionuclide is an α-emitter, more preferably is ²²⁵Ac and wherein preferably the activity dosage of the complex is at least 75 kBq/kg body, more preferably at least 100 kBq/kg body weight.
40. A PSMA binding ligand of any one of embodiments 1 to 33, a complex of embodiment 34 or 35, or a pharmaceutical composition of claim 36 for use in diagnostics.
41. A PSMA binding ligand of any one of embodiments for use of 1 to 33, a complex of embodiment 34 or 35, or a pharmaceutical composition of claim 36 for use in the diagnosis of cancer, preferably of PSMA expressing cancer, in particular of prostate cancer and/or metastases thereof.
42. The complex or the pharmaceutical composition for use of embodiment 40 or 41, wherein the radionuclide is a β-emitter, more preferably ¹⁷⁷Lu, and wherein preferably the activity dosage of the complex is at least 100 kBq/kg body weight, more preferably at least 500 kBq/kg body weight, most preferably at least 1 MBq/kg body weight.
43. A PSMA binding ligand of any one of embodiments for use of 1 to 33, a complex of embodiment 34 or 35, or a pharmaceutical composition of claim 36 for use in fluorescence guided surgery.
44. Method for identifying a tumor tissue to be removed by surgery++++, the method comprising detecting the presence of fluorescence in the tissue of a patient who was administered the PSMA binding ligand or the complex or a pharmaceutical composition of claim 23.
45. The PSMA ligand of embodiment 1 having one of the following structures:

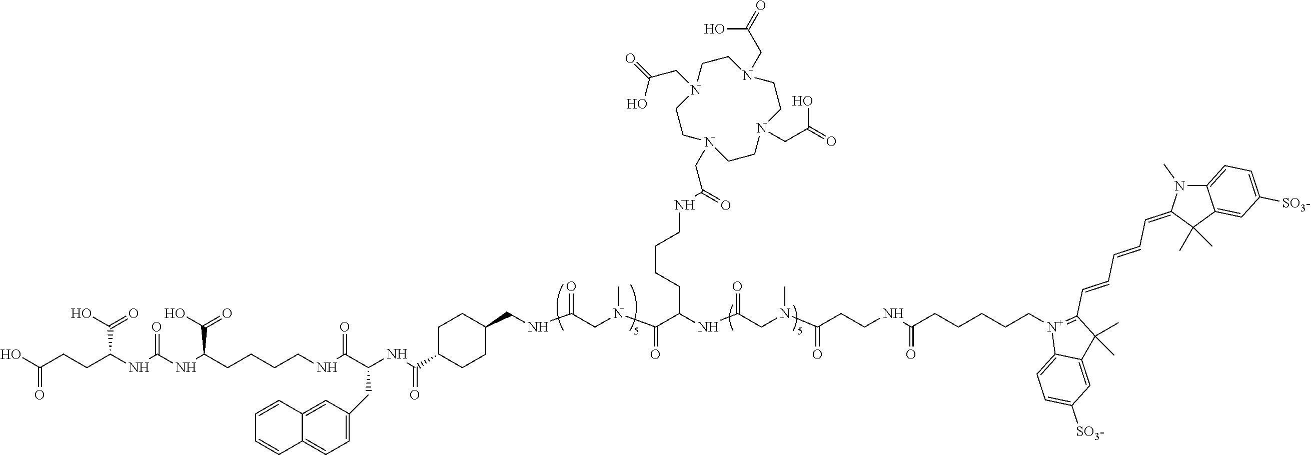

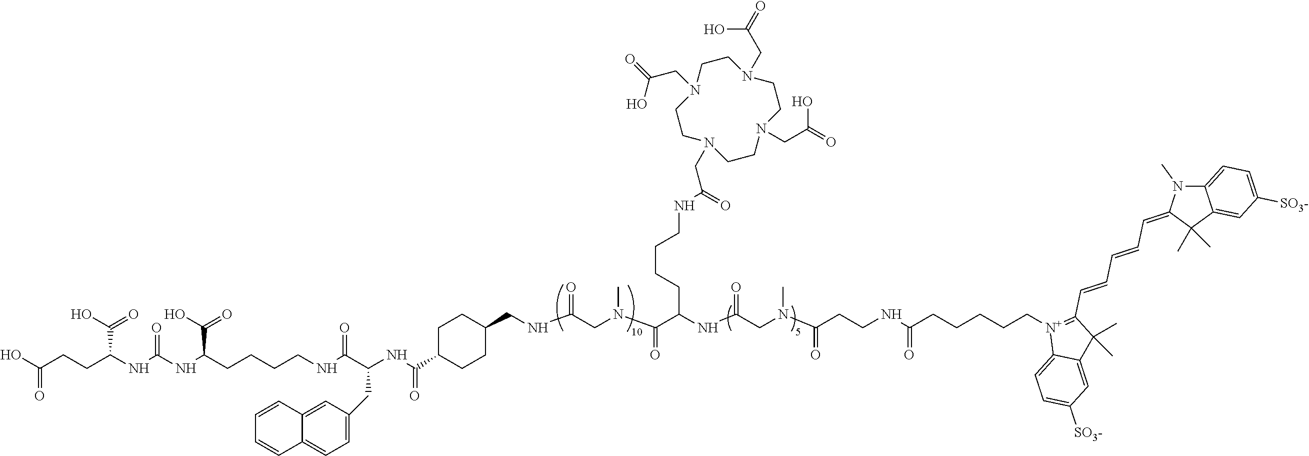

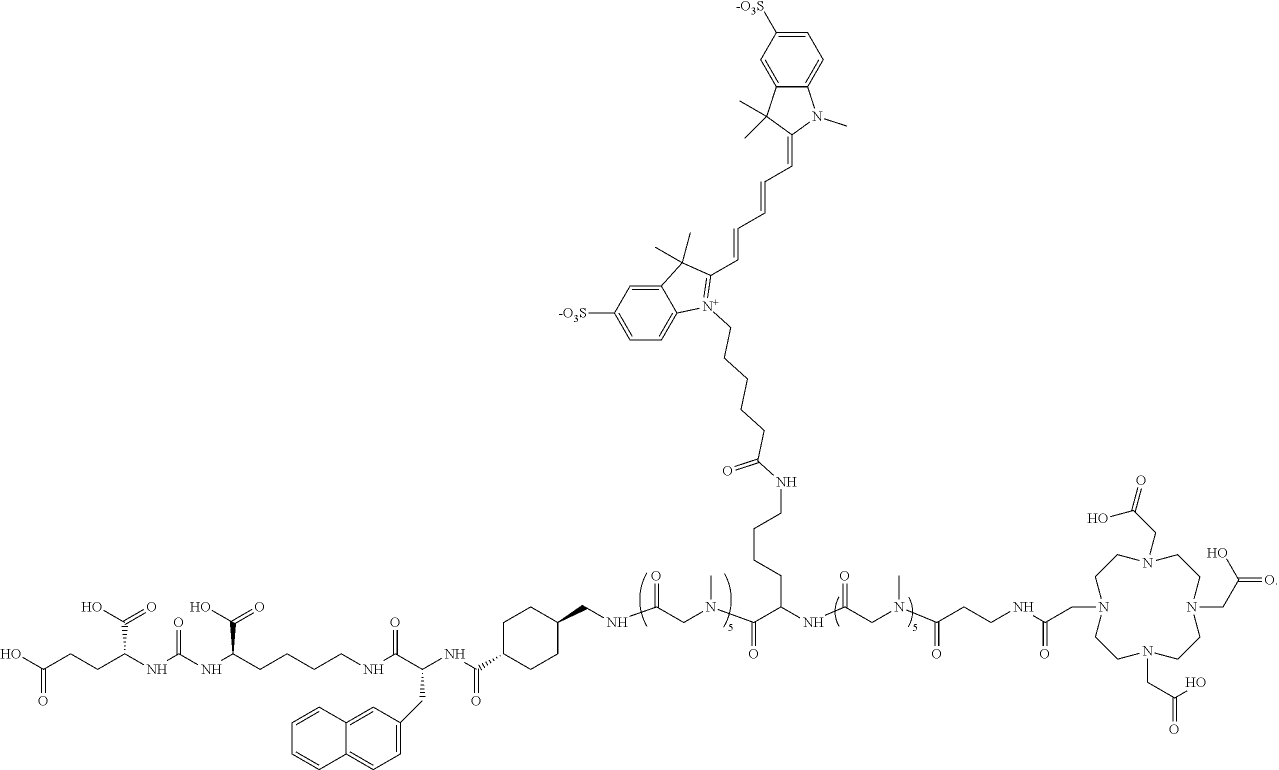

### FIGURE LEGENDS

FIG. 1: Structure of Glu-urea-Lys-2-Nal-Chx-Sar₅-Lys(DOTA)-Sar₅-bAla-sulfoCy5 (BP-1),

FIG. 2: Structure of Glu-urea-Lys-2-Nal-Chx-Sar₁₀-Lys(DOTA)-Sars-bAla-sulfoCy5 (BP-2)

FIG. 3: Structure of Glu-urea-Lys-2-Nal-Chx-Sar₅-Lys(sulfoCy5)-Sar₅-bAla-DOTA (BP-4)

FIG. 4a-c: Pharmacokinetic study with small-animal PET imaging. Time activity curves for non-target organs and tumor after injection of 0.5 nmol ⁶⁸Ga-labeled BP-2 in LNCaP- and PC-3 tumor-bearing athymic nude mice (right trunk) up to 60 min p.i. SUV=standardized uptake value.

FIG. 5: Optical imaging of tumor dissection. Optical imaging was performed after injection of 0.5 nmol ⁶⁸Ga-labeled BP-2 in LNCaP (A) and PC-3 (B) tumor-bearing BALB/c nu/nu mice (n=1). Mice were sacrificed 2 h p.i. after PET imaging and fluorescence detected with the Odyssey CLx system (excitation wavelength 700 nm). Fluorescence intensity is presented in heat map colouring. Tissue lying on the surface of the imaging system during fluorescence detection explains small artefacts in fluorescence images. The PSMA⁺-tumor showed a high fluorescence signal (FIG. 5A), while in the PSMA⁻-tumor (FIG. 5B) only a negligible fluorescence signal of BP-2 was detectable.

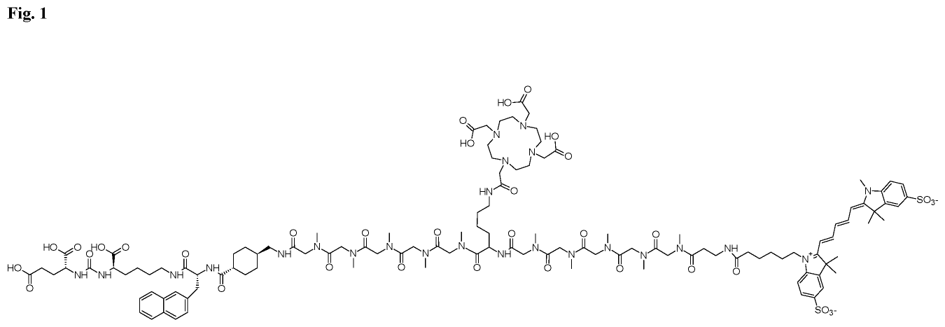

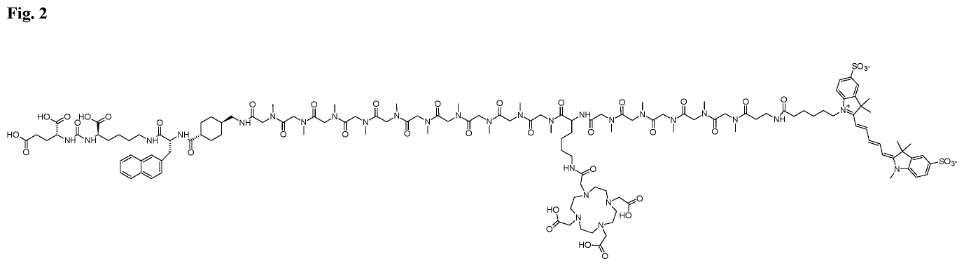

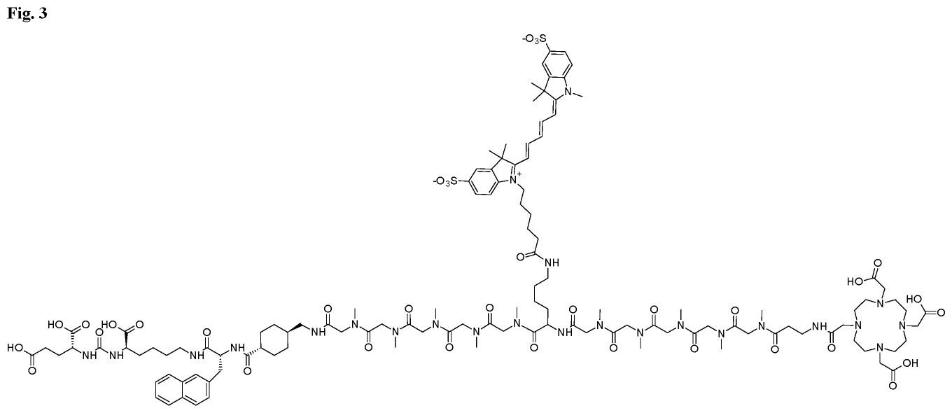

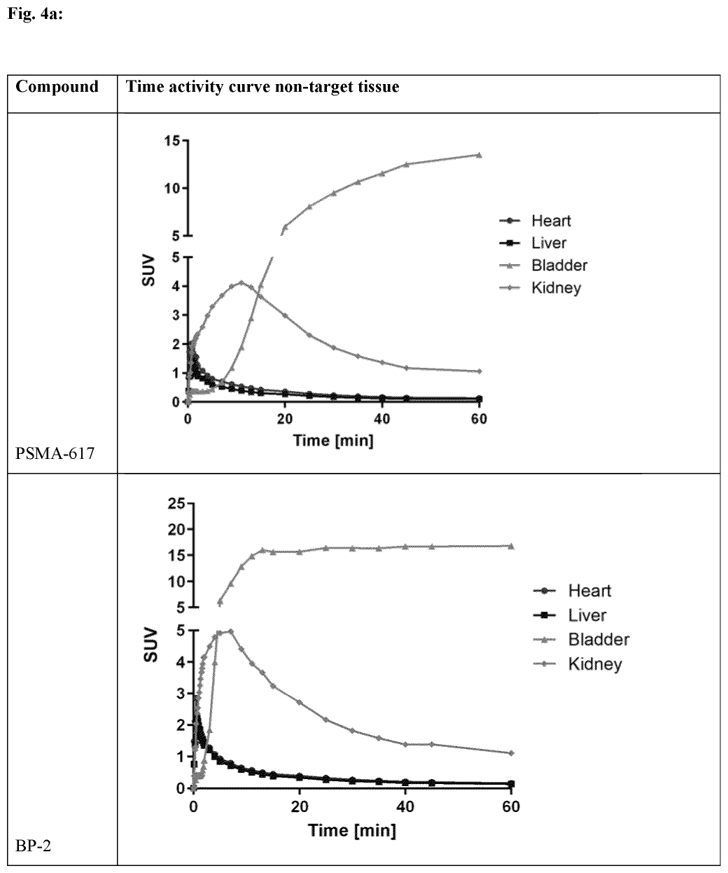

The following Examples shall merely illustrate the invention. They shall not be construed, whatsoever, to limit the scope of the invention.

### EXPERIMENTAL PROCEDURES

All commercially available chemicals were of analytical grade and used without further purification. ⁶⁸Ga (half-life 68 min) was obtained from a ⁶⁸Ge/⁶⁸Ga generator (Galliapharm® Ge-68/Ga-68 Generator, Eckert&Ziegler) and ¹⁷⁷Lu (half-life 6.6 d) was purchased from ITG. The compounds were purified using semipreparative reversed-phase high performance liquid chromatography (RP-HPLC; Chromolith Semi Prep RP-18e, 100×10 mm; Merck, Darmstadt, Germany). Compound analysis was performed using analytical RP-HPLC (RP-HPLC; Chromolith RP-18e, 100×4.6 mm; Merck, Darmstadt, Germany). Analytical HPLC runs were performed using a linear gradient (5% A (0.1% aqueous TFA) to 100% B (0.1% TFA in CH₃CN)) in 10 min at 2 mL/min. The system Agilent Technologies 1200 Series was equipped with a variable UV and a gamma detector (Ramona*, Elysia). UV absorbance was measured at 220 and 280 nm, respectively. For mass spectrometry a MALDI-MS (Daltonics Microflex, Bruker Daltonics, Bremen, Germany) was used.

### Synthesis of Glu-urea-Lys-2-Nal-Chx-Sar₅-Lys(DOTA)-Sar₅-bAla-sulfoCy5 (BP-1), Glu-urea-Lys-2-Nal-Chx-Sar₁₀-Lys(DOTA)-Sar₅-bAla-sulfoCy5 (BP-2) and Glu-urea-Lys-2-Nal-Chx-Sar₅-Lys(sulfoCy5)-Sar₅-bAla-DOTA (BP-4)

The synthesis of the pharmacophore Glu-urea-Lys was performed as described previously (1). Briefly, the synthesis started with the formation of the isocyanate of the glutamyl moiety using triphosgene. A resin-immobilized (2-chloro-tritylresin, Merck, Darmstadt) s-allyloxycarbonyl protected lysine was added and reacted for 16 h with gentle agitation. The resin was filtered off and the allyloxy-protecting group was removed by reacting twice with Pd(PPh₃)₄ (0.3 eq.) and morpholine (15 eq.) under ambient conditions (1 h, RT).

Subsequently, the linker between the PSMA pharmacophore and the chelator/dye was introduced by standard Fmoc solid phase protocol. In a first step Fmoc-2-NaI—OH and N-Fmoc-tranexamic acid (4 eq. each) with HATU (4 eq.) and DIPEA (10 eq.) were coupled in DMF. Depending on the amino acid sequence, Fmoc-sarcosine was coupled five and ten-times, respectively, followed by Fmoc-Lys(Alloc)-OH with HATU (4 eq.) and DIPEA (10 eq.) in DMF. Subsequently, Fmoc-sarcosine was coupled five-times followed by Boc-beta-Alanine with HATU (4 eq.) and DIPEA (10 eq.) in DMF.

For the synthesis of BP-1 and BP-2 the allyloxy-protecting group was removed by reacting twice with Pd(PPh₃)₄ (0.3 eq.) and morpholine (15 eq.) under ambient conditions (1 h, RT). Bis(tBu)DOTA (bis(tBu)-ester of 1,4,7,10-tetraazacyclododecan-1,4,7,10-tetraacetic acid) (4 eq.) with HATU (4 eq.) and DIPEA (10 eq.) was afterwards coupled in DMF. The precursors were cleaved from the resin for 3 hours at RT using TFA/TIPS/H₂O (95/2.5/2.5, v/v/v) and purified using RP-HPLC using a Chromolith RP-18e column (100×10 mm; Merck, Darmstadt, Germany) and identified with mass spectrometry. Finally, sulfoCy5-NHS ester (2.5 mg) was conjugated to the precursors in DMF for 24 h at RT and the final products (BP-1 and BP-2) purified using RP-HPLC using a Chromolith RP-18e column (100×10 mm; Merck, Darmstadt, Germany) and identified with mass spectrometry.

For the synthesis of BP-4, Bis(tBu)DOTA (bis(tBu)-ester of 1,4,7,10-tetraazacyclododecan-1,4,7,10-tetraacetic acid) (4 eq.) with HATU (4 eq.) and DIPEA (10 eq.) were coupled in DMF to Glu-urea-Lys-2-Nal-Chx-Sar₅-Lys(Alloc)-Sar₅-bAla. Afterwards, the allyloxy-protecting group was removed by reacting twice with Pd(PPh₃)₄ (0.3 eq.) and morpholine (15 eq.) under ambient conditions (1 h, RT). The precursor was cleaved from the resin for 3 hours at RT using TFA/TIPS/H₂O (95/2.5/2.5, v/v/v) and lyophilized. Finally, sulfoCy5-NHS ester (2.5 mg) was conjugated to the precursor in DMF for 24 h at RT and the final product (BP-4) purified using RP-HPLC using a Chromolith RP-18e column (100×10 mm; Merck, Darmstadt, Germany) and identified with mass spectrometry.

### ⁶⁸Ga—Labeling

The precursor peptide [5 nmol in HEPES buffer (1 M, pH 4, 40 μL)] was added to 40 μL [⁶⁸G]G³⁺ eluate (˜40 MBq) and ascorbic acid (5.68 mM, 0.8 μL). The pH was adjusted to 3.8-4.2 using 30% NaOH. The reaction mixture was incubated at 95° C. for 15 minutes. The radiochemical yield (RCY) was determined by RP-HPLC.

### ¹⁷⁷Lu—Labeling

The precursor peptide [2 nmol in HEPES buffer (0.1 M, pH 7, 50 μL)] was added to 10 μL [¹⁷⁷Lu]LuCl₃ (˜10-30 MBq, 0.04 M HCl) and ascorbic acid (5.68 mM, 0.7 μL). The reaction mixture was incubated at 95° C. for 15 minutes. The radiochemical yield (RCY) was determined by RP-HPLC.

### Cell Culture

PSMA⁺ LNCaP cells (CRL-1740; ATCC; PSMA-positive) and PC-3 cells (CRL-1435; ATCC; PSMA-negative) were cultured in RPMI medium supplemented with 10% fetal calf serum and 2 mmol/L L-glutamine (all from PAA). Cells were grown at 37° C. in humidified air with 5% CO₂ and were harvested using trypsin-ethylenediaminetetraacetic acid (trypsin-EDTA; 0.25% trypsin, 0.02% EDTA, Invitrogen).

### Cell Binding and Internalization

The competitive cell binding assay and internalization experiments were performed as described previously (2). Briefly, the cells (10⁵ per well) were incubated with a 0.8 nM solution of ⁶⁸Ga-labeled radioligand [Glu-urea-Lys(Ahx)]₂-HBED-CC (PSMA-10, precursor ordered from ABX, Radeberg, Germany) in the presence of 12 different concentrations of analyte (0-5000 nM, 100 μL/well). After incubation, the mixture was removed and the wells were washed 3 times with PBS using a multiscreen vacuum manifold (Millipore, Billerica, MA). Cell-bound radioactivity was measured using a gamma counter (Perkin Elmer 2480, Wizard, Gamma Counter). The 50% inhibitory concentration (IC50) values were calculated by fitting the data using a nonlinear regression algorithm (GraphPad Software).

For internalization experiments, 10⁵ cells per well were seeded in poly-L-lysine coated 24-well cell culture plates 24 h before incubation. After washing, the cells were incubated with 30 nM of the radiolabeled compound for 45 min at 37° C. Cellular uptake was terminated by washing 3 times with 1 mL of ice-cold PBS. To remove surface-bound radioactivity, cells were incubated twice with 0.5 mL glycine-HCl in PBS (50 mM, pH=2.8) for 5 min. The cells were washed with 1 mL of ice-cold PBS and lysed using 0.3 N NaOH (0.5 mL). The surface-bound and the internalized fractions were measured in a gamma counter. The cell uptake was calculated as percent of the initially added radioactivity bound to 10⁵ cells [% ID/10⁵ cells].

### Biodistribution

For the experimental tumor models 5×10⁶ cells of LNCaP or PC-3 (in 50% Matrigel; Becton Dickinson) were subcutaneously inoculated into the right trunk of 7- to 8-week-old male BALB/c nu/nu mice (Janvier). The tumors were allowed to grow until approximately 1 cm³ in size. The ¹⁷⁷Lu-labeled compounds were injected into a tail vein (1-2 MBq; 60 μmol). At 1 h or 2 h after injection (p.i.) the animals were sacrificed. Organs of interest were dissected, blotted dry, and weighed. The radioactivity was measured using a gamma counter and calculated as % ID/g. All animal experiments complied with the current laws of the Federal Republic of Germany.

### PET/MR and Optical Imaging

For imaging studies, mice were anesthetized (2% isoflurane) and 0.5 nmol of the ⁶⁸Ga-labeled compound in 0.9% NaCl (pH 7) were injected into the tail vein. PET imaging was performed with μPET/MRI scanner (BioSpec 3T, Bruker) with a dynamic scan for 60 min. The images were iteratively reconstructed (MLEM 0.5 algorithm, 12 iterations) and were converted to SUV images. Quantification was done using a ROI (region of interest) technique and data in expressed in time activity curves as SUV_{bodyweight}. Mice were sacrificed after PET/MR imaging and optical imaging of the subcutaneous tumor and organs of interest was performed with the Odyssey CLx system (LI-COR Biosciences, excitation wavelength 700 nm). All animal experiments complied with the current laws of the Federal Republic of Germany.

### Statistical Aspects

All experiments were performed at least in triplicate and repeated at least for three times. Quantitative data were expressed as mean±SD. If applicable, means were compared using Student's t test. P values <0.05 were considered statistically significant.

### Results

### In Vitro Characterization

The final product was identified using reversed-phase HPLC/matrix-assisted laser desorption/ionization mass spectrometry. The ⁶⁸Ga and ¹⁷⁷Lu complexation of all compounds resulted in radiochemical yields higher than 95%. All compounds showed a high PSMA-binding affinity in the nanomolar range, which was reduced as compared to the reference PSMA-617 (Table 1) (3). Remarkably, a PSMA-specific cell surface binding and specific internalization comparable to PSMA-617 was detected for all tested ⁶⁸Ga-labeled compounds.

TABLE 1
Cell binding and internalization data of the compounds*. Affinity
to PSMA and internalization properties of the compounds were determined
in vitro using PSMA⁺-cells (LNCaP). For all compounds
PSMA-specific internalization and a binding affinity to PSMA
in the nanomolar range were detected.

| | Specifically cell | Specifically | |
|---|---|---|---|
| | surface bound | internalized | kᵢ [nM]^{‡} |
| Compound | [% AR/10⁵ cells]^{†} | [% AR/10⁵ cells]^{†} | free ligands |
| BP-2 | 1.2 ± 0.8 | 0.5 ± 0.4 | 71.91 ± 21.18 |
| BP-1 | 1.3 ± 0.6 | 1.1 ± 1.0 | 62.43 ± 16.53 |
| BP-4 | 0.8 ± 1.0 | 0.4 ± 0.7 | 64.94 ± 6.82 |
| PSMA-617 | 0.3 ± 0.1 | 1.2 ± 0.6 | 2.3 ± 2.9 |

*Data are expressed as mean ± SD (n = 3),
^{†68}Ga-labeled compounds; kᵢ-value for PSMA-617 from Benesova et al. (3). Specific cell uptake was determined by blockage using 500 μM 2-PMPA. Values are expressed as % of applied radioactivity (AR) bound to 10⁵ cells.
^{‡}radioligand: ⁶⁸Ga-PSMA-10 (K_{d}: 3.8 ± 1.8 nM (1), c_{radioligand}: 0.8 nM)

### In Vivo Characterization

All tested ¹⁷⁷Lu-labeled compounds revealed a PSMA-specific tumor uptake in LNCaP xenograft tumors (p>0.05), which is not significantly different compared to reference compound ¹⁷⁷Lu-PSMA-617 at 1 h p.i. (8.47±4.09% ID/g) and ⁶⁸Ga-Glu-urea-Lys-2-Nal-Chx-Lys(IRDye800CW)-DOTA (4.13±0.15% ID/g) (Table 2) (4). At the same time, the kidney uptake of 177Lu—BP-2 (19.05±9.52% ID/g) was surprisingly significantly reduced compared to ¹⁷⁷Lu-PSMA-617 (137.2±77.8% ID/g), ⁶⁸Ga-Glu-urea-Lys-2-Nal-Chx-Lys(IRDye800CW)-DOTA (65.64±6.60% ID/g) and the other tested compounds ¹⁷⁷Lu—BP-1 and ¹⁷⁷Lu—BP-4. The uptake of BP-2 in muscle, blood, spleen, lung and liver was found to be comparable to the other tested compounds at 1 h p.i. (Table 3). Fast renal clearance is confirmed by further decreased BP-2 uptake found in the kidney (4.61±0.45% ID/g) accompanied by decreased uptake in e.g. blood and spleen at 2 h p.i. The tumor uptake differs not significantly at 2 h p.i. as compared to 1 h p.i., indicating tracer enrichment over time. Tumor specificity of BP-2 was proven in PC-3 tumor-bearing mice, showing only negligible tumor uptake at 1 h p.i. (Table 2).

TABLE 2
Organ distribution of 0.06 nmol ¹⁷⁷Lu-labeled compounds in tumor bearing BALB/c nu/nu mice 1 h*.

| | | ¹⁷⁷Lu-BP-2 | | ¹⁷⁷Lu-BP-1 | | ¹⁷⁷Lu-BP-4 |
|---|---|---|---|---|---|---|
| | ¹⁷⁷Lu-BP-2 | LNCaP Tumor- | ¹⁷⁷Lu-BP-1 | LNCaP Tumor- | ¹⁷⁷Lu-BP-4 | LNCaP Tumor- |
| | [% ID/g] | to-Organ ratio | [% ID/g] | to-Organ ratio | [% ID/g] | to-Organ ratio |
| Blood | 0.16 ± 0.01 | 34.73 ± 14.72 | 0.16 ± 0.02 | 39.99 ± 4.75 | 0.23 ± 0.08 | 25.89 ± 9.14 |
| Heart | 0.07 ± 0.01 | 90.47 ± 45.17 | 0.10 ± 0.004 | 64.45 ± 12.77 | 0.12 ± 0.02 | 48.59 ± 15.65 |
| Lung | 0.27 ± 0.01 | 19.88 ± 6.95 | 0.38 ± 0.03 | 18.17 ± 4.54 | 0.40 ± 0.04 | 12.10 ± 2.03 |
| Spleen | 1.65 ± 0.86 | 6.35 ± 6.16 | 0.78 ± 0.03 | 8.05 ± 1.05 | 0.59 ± 0.12 | 7.83 ± 0.85 |
| Liver | 1.18 ± 0.61 | 7.93 ± 7.53 | 0.24 ± 0.03 | 27.78 ± 5.94 | 0.14 ± 0.01 | 36.13 ± 6.70 |
| Kidney | 8.49 ± 3.38 | 0.66 ± 0.20 | 18.86 ± 4.56 | 0.34 ± 0.04 | 37.50 ± 12.13 | 0.17 ± 0.08 |
| Muscle | 0.03 ± 0.01 | 186.23 ± 31.89 | 0.04 ± 0.004 | 165.23 ± 31.45 | 0.06 ± 0.002 | 110.15 ± 51.69 |
| Intestine | 0.10 ± 0.06 | 83.92 ± 53.16 | 0.08 ± 0.01 | 76.31 ± 13.13 | 0.12 ± 0.04 | 49.30 ± 18.19 |
| Brain | 0.02 ± 0.005 | 286.72 ± 78.41 | 0.02 ± 0.001 | 423.06 ± 81.33 | 0.02 ± 0.002 | 275.08 ± 63.60 |
| Tumor | 5.38 ± 2.03 | — | 5.85 ± 1.16 | — | 4.30 ± 0.27 | — |
| LNCaP | | | | | | |
| Tumor | 0.37 ± 0.03 | | — | — | — | — |
| PC-3 | | | | | | |

*Data are expressed as mean % ID/g tissue ± SD (n = 3, for BP-2 n = 4) and Tumor-to-Organ ratios

TABLE 3
Organ distribution of 0.06 nmol ¹⁷⁷Lu-labeled BP-2
in LNCaP-tumor bearing BALB/c nu/nu mice 2 h p.i.*.

| | ¹⁷⁷Lu-BP-2 | ¹⁷⁷Lu-BP-2 |
|---|---|---|
| | [% ID/g] | Tumor-to-Organ ratio |
| Blood | 0.05 ± 0.01 | 88.59 ± 20.90 |
| Heart | 0.03 ± 0.003 | 124.71 ± 13.31 |
| Lung | 0.12 ± 0.02 | 36.31 ± 5.39 |
| Spleen | 1.37 ± 0.10 | 3.10 ± 0.24 |
| Liver | 1.25 ± 0.13 | 3.41 ± 0.27 |
| Kidney | 4.61 ± 0.45 | 0.93 ± 0.14 |
| Muscle | 0.06 ± 0.07 | 181.13 ± 119.38 |
| Intestine | 0.03 ± 0.004 | 147.74 ± 23.30 |
| Brain | 0.01 ± 0.003 | 419.27 ± 102.70 |
| Tumor | 4.23 ± 0.19 | — |

*Data are expressed as mean % ID/g tissue ± SD (n = 3) and Tumor-to-Organ ratios

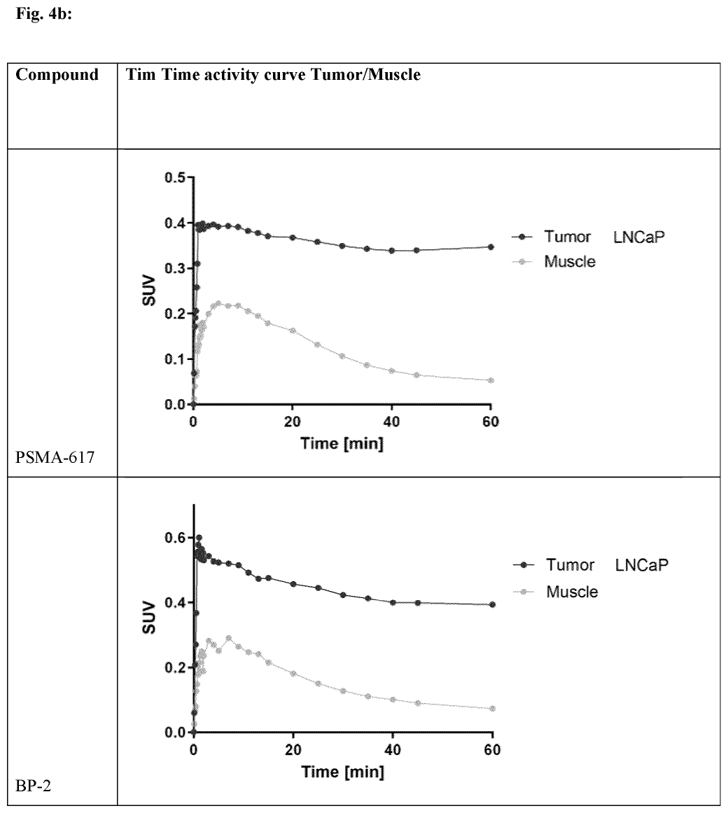

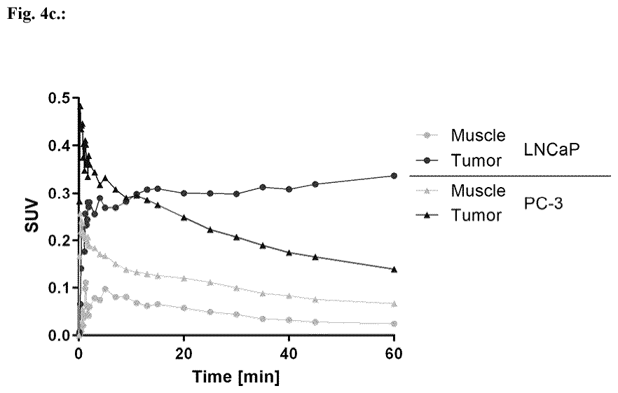

The organ distribution findings were confirmed by PET/MR imaging. The pharmacokinetic properties of BP-2 are enhanced compared to PSMA-617 characterized by an accelerated excretion profile. The tumor targeting properties of BP-2 were found to be comparable to the parental reference PSMA-617 (FIG. 4).

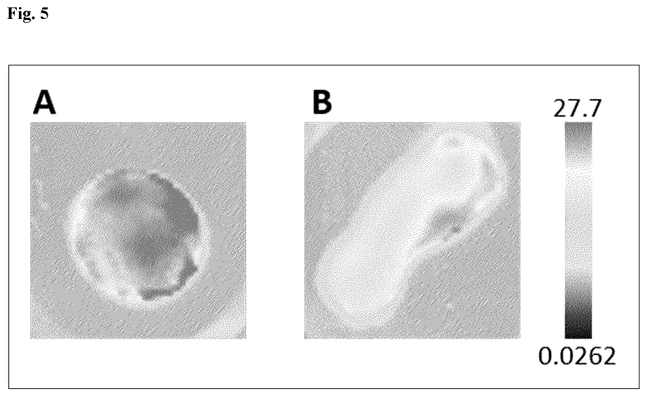

Optical imaging confirmed the findings of PSMA-specific tumor enrichment (FIG. 5). Here, the PSMA⁺-tumor showed a high fluorescence signal (FIG. 5A), while in the PSMA⁻-tumor (FIG. 5B) only a negligible fluorescence signal of BP-2 was detectable.

### REFERENCES

1. Schafer M, Bauder-Wust U, Leotta K, et al. A dimerized urea-based inhibitor of the prostate-specific membrane antigen for 68Ga-PET imaging of prostate cancer. EJNMVII Res. 2012; 2:23.
2. Eder M, Schafer M, Bauder-Wust U, et al. (68)Ga-Complex lipophilicity and the targeting property of a urea-based PSMA inhibitor for PET imaging. Bioconjug Chem. 2012; 23:688-697.
3. Benesova M, Bauder-Wust U, Schafer M, et al. Linker Modification Strategies To Control the Prostate-Specific Membrane Antigen (PSMA)-Targeting and Pharmacokinetic Properties of DOTA-Conjugated PSMA Inhibitors. J Med Chem. 2016; 59:1761-1775.
4. Benesova M, Schafer M, Bauder-Wust U, et al. Preclinical Evaluation of a Tailor-Made DOTA-Conjugated PSMA Inhibitor with Optimized Linker Moiety for Imaging and Endoradiotherapy of Prostate Cancer. J Nucl Med. 2015; 56:914-920.

1-15. (canceled)

16. A PSMA binding ligand or a pharmaceutically acceptable salt or solvate thereof comprising a PSMA binding motif Q, a chelator residue A, a dye group Z and at least one linker L^{BQ} comprising at least one amino acid X₁, preferably at least one N-alkylated amino acid, more preferably wherein the amino acid X₁ is —N(CH₃)—CH₂—C(═O)—.

17. The PSMA binding ligand of claim 16 having the structure: 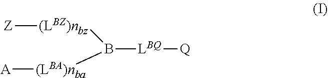
wherein:
Q is the PSMA binding motif,
A is the chelator residue,
Z is the dye group,
B is a branching group,
L^{BQ} is a linker connecting Q with B, wherein the linker comprises the at least one amino acid X₁,
L^{BZ} is a linker, wherein n_{bz} is 0 or 1, and
L^{BA} is a linker, wherein n_{ba} is 0 or 1.

18. The PSMA binding ligand of claim 17, wherein B has the structure: 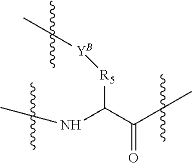
wherein:
R₅ is an alkyl group, preferably a —(CH₂)₂₋₄ group, and
Y^{B} is a functional group linking R₅ to the group -(L^{BZ})n_{bz}-Z or -(L^{BA})n_{ba}-A, wherein Y^{B} is preferably a group —NH— or —C(═O)—.

19. The PSMA binding ligand of claim 18, having one of the following structures (Ia) or (Ib), preferably the structure (Ia):

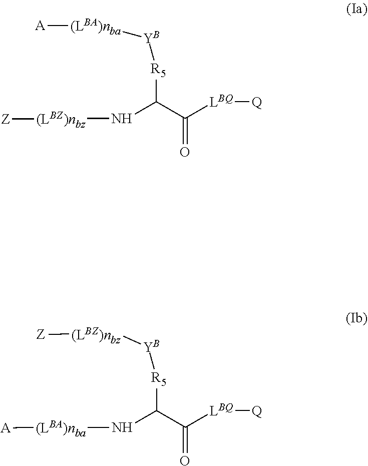

20. The PSMA binding ligand of claim 16 or a pharmaceutically acceptable salt or solvate thereof,
wherein A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid](NOPO), 3,6,9,15-tetraazabicyclo[9.3.1]pentadeca-1 (15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA).

21. The PSMA binding ligand of claim 16 or a pharmaceutically acceptable salt or solvate thereof, wherein A is a chelator residue having the structure:

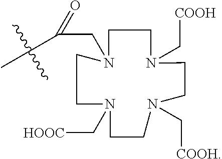 [SMILES: *C(=O)CN1CCN(CC(=O)O)CCN(CC(=O)O)CCN(CC(=O)O)CC1]

22. The PSMA ligand of claim 17, wherein the dye group Z is a fluorescent dye Z comprising, preferably consisting of, the structure: 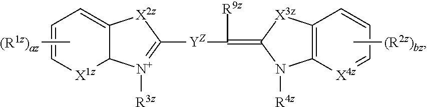
wherein:
X^{1z} and X^{4z} are independently selected from the group consisting of —N=, —N(R^{5z})=, and —C(R^{6z})═;
X^{2z} and X^{3z} are independently selected from the group consisting of O, S, Se, N(R^{Sz}), and C(R^{6z}R^{7z}), preferably both are C(CH₃)₂;
Y^{z} is a linker connecting the two moieties of (C) and permitting electron delocalization between said moieties, wherein Y^{z} optionally comprises a group 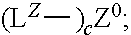
az and bz are independently selected from the group consisting of 1, 2, and 3;
each R^{1z} and each R^{2z} is independently selected from the group consisting of (L^{z}-)_{c}Z^{z}, (L^{z}-)_{c}Z⁰ and H; and two adjacent R^{1z} and/or two adjacent R^{2z} can also form an aromatic ring, which is optionally substituted with one or more (L^{z}-)_{c}Z^{z} or (L^{z}-)_{c}Z⁰;
R^{3z}, R^{4z}, R^{5z}, R^{6z}, R^{7z}, R^{9z} are independently selected from the group consisting of (L^{z}-)_{c}Z^{z}, (L^{z}-)_{c}Z⁰, and H;
each c is independently 0 or 1;
each L^{z} is independently T¹, —OT¹-, —ST¹-, —C(O)T¹-, —C(O)OT¹-, —OC(O)T¹-, —C(O)NHT¹-, —NHC(O)T¹, or a C₁₋₁₀ alkylene group, which is optionally interrupted and/or terminated by one or more of —O—, —S—, —C(O)—, —C(O)O—, —OC(O)—, —C(O)NH—, —NHC(O)O—, and T¹;
T¹ is phenyl, naphthyl, indenyl, indanyl, tetralinyl, decalinyl, adamantyl, C₃₋₇ cycloalkyl, 3 to 7 membered heterocyclyl, or 7 to 11 membered heterobicyclyl, wherein T¹ is optionally substituted with one or more substituents selected from the group consisting of halogen, CN, C(O)R^{8z}, COOR^{8z}, OR^{8z}, C(O)N(R^{8z}R^{8az}), S(O)₂N(R^{8z}R^{8az}), S(O)N(R^{8z}R^{8az}), S(O)₂R^{8z}, N(R^{8z})S(O)₂N(R^{8az}R^{8bz}), SR^{8z}, N(R^{8z}R^{8az}), NO₂; OC(O)R^{8z}, N(R^{8z})C(O)R^{8az}, N(R^{8z})S(O)₂R^{8az}, N(R^{8z})S(O)R^{8az}, N(R^{8z})C(O)N(R^{8az}R^{8bz}), N(R^{8z})C(O)OR^{8az}, OC(O)N(R^{8z}R^{8az}), oxo (═O), where the ring is at least partially saturated, or C₁₋₆ alkyl, wherein C₁₋₆ alkyl is optionally substituted with one or more halogen, which are the same or different;
each Z^{z} is independently H, halogen, CN, C(O)R^{8z}, C(O)OR^{8z}, C(O)O⁻ OR^{8z}, C(O)N(R^{8z}R^{8az}), S(O)₂OR^{8z}, S(O)₂O⁻, S(O)₂N(R^{8z}R^{8az}), S(O)N(R^{8z}R^{8az}), S(O)₂R^{8z}, S(O)R^{8z}, N(R^{z})S(O)₂N(R^{8az}R^{8bz}), SR^{8z}, N(R^{8z}R^{8az}), NO₂; P(O)(OR^{8z})₂, P(O)(OR^{8z})O⁻, OC(O)R^{8z}, N(R^{8z})C(O)R^{8az}, N(R^{8z})S(O)₂R^{8az}, N(R^{8z})S(O)R^{8z}, N(R^{8z})C(O)N(R^{8az}R^{8bz}), N(R^{8z})C(O)OR^{8az}, or OC(O)N(R^{8z}R^{8az});
R^{8z}, R^{8az}, R^{8bz} are independently selected from the group consisting of H and C₁₋₆ alkyl, wherein C₁₋₆ alkyl is optionally substituted with one or more halogen which are the same or different;
Z⁰ is a chemical bond connecting the dye Z to the group -(L^{BZ})n_{bz}-B, provided that one of R^{1z}, R^{2z}, R^{3z}, R^{4z}, R^{5z}, R^{6z}, R^{7z}, R^{9z} is (L^{z}-)_{c}Z⁰ or that Y^{z} comprises (L-)_{c}Z⁰.

23. The PSMA binding ligand of claim 16, wherein the ligand has the structure (IIIa): 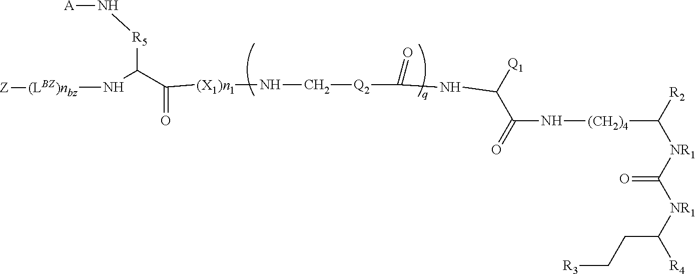
wherein:
R1 is H or —CH₃, preferably H,
R2, R3 and R4 are independently of each other, selected from the group consisting of—CO₂H, —SO₂H, —SO₃H, —OSO₃H, —PO₂H, —PO₃H and —OPO₃H₂,
Q1 is selected from the group consisting of alkylaryl, arylalkyl, aryl, alkylheteroaryl, heteroarylalkyl and heteroaryl,
Q2 is selected from the group consisting of aryl, alkylaryl, arylalkyl, cycloalkyl, heterocycloalkyl, heteroaryl, heteroarylalkyl and alkylheteroaryl,
q is an integer of from 0-3,
X1 is a methylated amino acid, preferably wherein X1 is —N(CH₃)—CH₂—C(═O)— and n1 is an integer of from 1 to 25, preferably 5 to 10, more preferably 10,
Z is the dye group,
L^{BZ} is a linker, wherein nbz is 0 or 1, and
R5 is an alkyl group, preferably a —(CH2)2-4 group.

24. A complex comprising:
(a) a radionuclide, and
(b) the PSMA binding ligand or a pharmaceutically acceptable salt or solvate thereof of claim 16.

25. The complex of claim 24, wherein the radionuclide is selected from the group consisting of ⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb), more preferably selected from the group consisting of ⁹⁰Y, ⁶⁸Ga, ¹⁷⁷Lu, ²²⁵Ac, and ²¹³Bi, more preferably the radionuclide is ¹⁷⁷Lu or ²²⁵Ac.

26. A pharmaceutical composition comprising the PSMA binding ligand or a pharmaceutically acceptable salt or solvate thereof of claim 16.

27. A method for treating and/or preventing PSMA expressing cancer and/or metastases thereof, in particular prostate cancer and/or metastases thereof, comprising administering to a subject in need the complex of claim 24.

28. A method for treating and/or preventing PSMA expressing cancer and/or metastases thereof, in particular prostate cancer and/or metastases thereof, comprising administering to a subject in need the PSMA binding ligand or a pharmaceutically acceptable salt or solvate thereof of claim 16.

29. The method of claim 27, wherein adverse side effects on the kidney are reduced and/or avoided.

30. The method of claim 28, wherein adverse side effects on the kidney are reduced and/or avoided.

31. A method for diagnosing cancer and/or metastases thereof, preferably PSMA-expressing cancer and/or metastases thereof, in particular prostate cancer and/or metastases thereof, comprising using a diagnostic method comprising the complex of claim 24.
